(19) United States
(12) Patent Application Publication
(10) Pub. No.: US 2023/0000321 A1
(43) Pub. Date: Jan. 5, 2023

(54) OPTICAL IMAGING SYSTEM

(71) Applicant: Gentuity, LLC, Sudbury, MA (US)

(72) Inventors: Giovanni J. Ughi, Arlington, MA (US); Christopher Battles, Seymour, CT (US); Christopher C. Petroff, Groton, MA (US); Christopher L. Petersen, Carlisle, MA (US)

(21) Appl. No.: 17/682,197

(22) Filed: Feb. 28, 2022

Related U.S. Application Data
(60) Provisional application No. 63/154,934, filed on Mar. 1, 2021.

(51) Int. Cl. A61B 1/00; A61B 1/06

(52) U.S. Cl. CPC A61B 1/00128; A61B 1/0676; A61B 1/00105; A61B 1/00009

(57) ABSTRACT

Provided herein are imaging systems for a patient including an imaging probe and an imaging assembly. The imaging probe includes: an elongate shaft with a proximal end, a distal portion, and a lumen extending between the proximal end and the distal portion; a rotatable optical core with a proximal end and a distal end, and at least a portion of the rotatable optical core is positioned within the lumen of the elongate shaft; and an optical assembly positioned proximate the distal end of the rotatable optical core, the optical assembly configured to direct light to tissue to be imaged and collect reflected light from the tissue to be imaged. The imaging assembly is constructed and arranged to optically couple to the imaging probe. The imaging assembly is configured to emit light into the imaging probe and receive the reflected light collected by the optical assembly.

### RELATED APPLICATIONS

This application claims the benefit of U.S. Provisional Application Ser. No. 63/154,934 (Docket No. GTY-021-PR1), titled “Optical Imaging System”, filed Mar. 1, 2021, the content of which is incorporated by reference in its entirety.

This application is related to U.S. Provisional Application Ser. No. 62/148,355 (Docket No.: GTY-001-PR1), titled “Micro-Optic Probes for Neurology”, filed Apr. 16, 2015, the content of which is incorporated by reference in its entirety.

This application is related to U.S. Provisional Application Ser. No. 62/322,182 (Docket No. GTY-001-PR2), titled “Micro-Optic Probes for Neurology”, filed Apr. 13, 2016, the content of which is incorporated by reference in its entirety.

This application is related to International PCT Patent Application Serial Number PCT/US2016/027764 (Docket No. GTY-001-PCT), titled “Micro-Optic Probes for Neurology” filed Apr. 15, 2016, Publication Number WO 2016/168605, published Oct. 20, 2016, the content of which is incorporated by reference in its entirety.

This application is related to U.S. patent application Ser. No. 15/566,041 (Docket No. GTY-001-US), titled “Micro-Optic Probes for Neurology”, filed Oct. 12, 2017, United States Publication Number 2018-0125372, published May 10, 2018, the content of which is incorporated by reference in its entirety.

This application is related to U.S. patent application Ser. No. 17/668,757 (Docket No. GTY-001-US-CON1), titled “Micro Optic Probes for Neurology”, filed Feb. 10, 2022, United States Publication Number ______, published ______, the content of which is incorporated by reference in its entirety.

This application is related to U.S. Provisional Application Ser. No. 62/212,173 (Docket No. GTY-002-PR1), titled “Imaging System Includes Imaging Probe and Delivery Devices”, filed Aug. 31, 2015, the content of which is incorporated by reference in its entirety.

This application is related to U.S. Provisional Application Ser. No. 62/368,387 (Docket No. GTY-002-PR2), titled “Imaging System Includes Imaging Probe and Delivery Devices”, filed Jul. 29, 2016, the content of which is incorporated by reference in its entirety.

This application is related to International PCT Patent Application Serial Number PCT/US2016/049415 (Docket No. GTY-002-PCT), titled “Imaging System Includes Imaging Probe and Delivery Devices”, filed Aug. 30, 2016, Publication Number WO 2017/040484, published Mar. 9, 2017, the content of which is incorporated by reference in its entirety.

This application is related to U.S. patent application Ser. No. 15/751,570 (Docket No. GTY-002-US), titled “Imaging System Includes Imaging Probe and Delivery Devices”, filed Feb. 9, 2018, U.S. Pat. No. 10,631,718, issued Apr. 28, 2020, the content of which is incorporated by reference in its entirety.

This application is related to U.S. patent application Ser. No. 16/820,991 (Docket No. GTY-002-US-CON1), titled “Imaging System Includes Imaging Probe and Delivery Devices”, filed Mar. 17, 2020 U.S. Pat. No. 11,064,873, issued Jul. 20, 2021, the content of which is incorporated by reference in its entirety.

This application is related to U.S. patent application Ser. No. 17/350,021 (Docket No. GTY-002-US-CON2), titled “Imaging System Includes Imaging Probe and Delivery Devices”, filed Jun. 17, 2021, Publication Number ______, published ______, the content of which is incorporated by reference in its entirety.

This application is related to U.S. Provisional Application Ser. No. 62/591,403 (Docket No. GTY-003-PR1), titled “Imaging System”, filed Nov. 28, 2017, the content of which is incorporated by reference in its entirety.

This application is related to U.S. Provisional Application Ser. No. 62/671,142 (Docket No. GTY-003-PR2), titled “Imaging System”, filed May 14, 2018, the content of which is incorporated by reference in its entirety.

This application is related to International PCT Patent Application Serial Number PCT/US2018/062766 (Docket No. GTY-003-PCT), titled “Imaging System”, filed Nov. 28, 2018, Publication Number WO 2019/108598, published Jun. 6, 2019, the content of which is incorporated by reference in its entirety.

This application is related to U.S. patent application Ser. No. 16/764,087 (Docket No. GTY-003-US), titled “Imaging System”, filed May 14, 2020, Publication Number 2020-0288950, published Sep. 17, 2020, the content of which is incorporated by reference in its entirety.

This application is related to U.S. Provisional Application Ser. No. 62/732,114 (Docket No. GTY-004-PR1), titled “Imaging System with Optical Pathway”, filed Sep. 17, 2018, the content of which is incorporated by reference in its entirety.

This application is related to International PCT Patent Application Serial Number PCT/US2019/051447 (Docket No. GTY-004-PCT), titled “Imaging System with Optical Pathway”, filed Sep. 17, 2019, Publication Number WO 2020/061001, published Mar. 26, 2020, the content of which is incorporated by reference in its entirety.

This application is related to U.S. patent application Ser. No. 17/276,500 (Docket No. GTY-004-US), filed Mar. 16, 2021, titled “Imaging system with Optical Pathway”, Publication Number 2021-0267442, published Sep. 2, 2021, the content of which is incorporated by reference in its entirety.

This application is related to U.S. Provisional Application Ser. No. 63/017,258 (Docket No. GTY-005-PR1), titled “Imaging System”, filed Apr. 29, 2020, the content of which is incorporated by reference in its entirety.

This application is related to International PCT Patent Application Serial Number PCT/US2021/29836 (Docket No. GTY-005-PCT), titled “Imaging System”, filed Apr. 29, 2021, Publication Number WO 2021/222530, published Nov. 4, 2021, the content of which is incorporated by reference in its entirety.

This application is related to U.S. Provisional Application Ser. No. 62/840,450 (Docket No. GTY-011-PR1), titled “Imaging Probe with Fluid Pressurization Element”, filed Apr. 30, 2019, the content of which is incorporated by reference in its entirety.

This application is related to International PCT Patent Application Serial Number PCT/US2020/030616 (Docket No. GTY-011-PCT), titled “Imaging Probe with Fluid Pressurization Element”, filed Apr. 30, 2020, Publication Number WO 2020/223433, published Nov. 5, 2020, the content of which is incorporated by reference in its entirety.

This application is related to U.S. patent application Ser. No. 17/600,212 (Docket No. GTY-011-US), titled “Imaging Probe with Fluid Pressurization Element”, filed Sep. 30, 2021, Publication Number ______, published ______, the content of which is incorporated by reference in its entirety.

This application is related to U.S. Provisional Application Ser. No. 62/850,945 (Docket No. GTY-013-PR1), titled “OCT-Guided Treatment of a Patient”, filed May 21, 2019, the content of which is incorporated by reference in its entirety.

This application is related to U.S. Provisional Application Ser. No. 62/906,353 (GTY-013-PR2), titled “OCT-Guided Treatment of a Patient”, filed Sep. 26, 2019, the content of which is incorporated by reference in its entirety. This application is related to International PCT Patent Application Serial Number PCT/US2020/033953 (Docket No. GTY-013-PCT), titled “Systems and Methods for OCT-Guided Treatment of a Patient”, filed May 21, 2020, Publication Number WO 2020/237024, published Nov. 26, 2020, the content of which is incorporated by reference in its entirety.

This application is related to U.S. patent application Ser. No. 17/603,689 (Docket No. GTY-013-US), titled “Systems and Methods for OCT-Guided Treatment of a Patient”, filed Oct. 14, 2021, Publication Number ______, published ______, the content of which is incorporated by reference in its entirety.

This application is related to U.S. Provisional Application Ser. No. 63/298,086 (Docket No. GTY-022-PR1), titled “Imaging System for Calculating Fluid Dynamics”, filed Jan. 10, 2022, the content of which is incorporated by reference in its entirety.

### FIELD OF THE INVENTION

The present invention relates generally to imaging systems, and in particular, intravascular imaging systems including imaging probes and delivery devices.

### BACKGROUND

Imaging probes have been commercialized for imaging various internal locations of a patient, such as an intravascular probe for imaging a patient's heart. Current imaging probes are limited in their ability to reach certain anatomical locations due to their size and rigidity. Current imaging probes are inserted over a guidewire, which can compromise their placement and limit use of one or more delivery catheters through which the imaging probe is inserted. There is a need for imaging systems that include probes with reduced diameter and high flexibility, as well as systems with one or more delivery devices compatible with these improved imaging probes.

### SUMMARY

According to an aspect of the present inventive concepts, an imaging system for a patient comprises an imaging probe, comprising: an elongate shaft comprising a proximal end, a distal portion, and a lumen extending between the proximal end and the distal portion; a rotatable optical core comprising a proximal end and a distal end, wherein at least a portion of the rotatable optical core is positioned within the lumen of the elongate shaft; and an optical assembly positioned proximate the distal end of the rotatable optical core, the optical assembly configured to direct light to tissue to be imaged and to collect reflected light from the tissue; and an imaging assembly constructed and arranged to optically couple to the imaging probe, the imaging assembly configured to emit light into the imaging probe and receive the reflected light collected by the optical assembly. The reflected light comprises image data, and the system is constructed and arranged to produce one or more images based on the image data. The imaging assembly can comprise multiple reference paths, and each reference path can comprise an optical fiber that comprises a different optical dispersion. The imaging assembly can be constructed and arranged to select a reference path that matches the optical dispersion of the rotatable optical core

In some embodiments, the elongate shaft further comprises an optically transparent window including one or more imagable portions, and the system further comprises an algorithm configured to utilize image data provided by the one or more imagable portions to reduce the negative impact of NURD on the one or more produced images.

In some embodiments, the system further comprises an algorithm, and the algorithm is configured to modify different image data portions of the one or more produced images based on one, two, or more characteristics of those image data portions. The algorithm can be configured to exponentially increase the intensity of an image data portion based on the distance of the portion from the center of the produced image. The algorithm can be configured to compensate the image data portion based on the physical, optical, and or other properties of the imaging system.

In some embodiments, the system further comprises a light source, and the imaging assembly is constructed and arranged to receive light from the light source, and the imaging assembly is constructed and arranged to duplicate and shift the light received from the light source. The light emitted into the imaging probe can comprise a duty cycle that is two times the duty cycle of the light received from the light source.

In some embodiments, the imaging probe further comprises a spring tip comprising a varying flexibility along its length, and during a pullback procedure, the spring tip can be constructed and arranged to remain distal to a pullback starting location of the optical assembly.

In some embodiments, the imaging assembly comprises multiple reference paths, and each reference path comprises an optical fiber that comprises a different optical dispersion, and the imaging assembly is constructed and arranged to select a reference path that matches the optical dispersion of the rotatable optical core.

In some embodiments, the optical assembly comprises a lens and an air-filled space distal to the lens, and the air-filled space is sealed with a porous plug comprising a sintered construction.

In some embodiments, the imaging probe comprises a centrifugal breaking assembly constructed and arranged to prevent the optical assembly from rotating above a threshold rate of rotation. The centrifugal breaking assembly can comprise an unbalanced centrifugal breaking assembly. The centrifugal breaking assembly can comprise a balanced centrifugal breaking assembly.

In some embodiments, the system further comprises a pullback module comprising a unidirectional locking mechanism constructed and arranged to frictionally engage the elongate shaft of the imaging probe, and a lead screw mechanism constructed and arranged to pull back the imaging probe via the locking mechanism.

The technology described herein, along with the attributes and attendant advantages thereof, will best be appreciated and understood in view of the following detailed description taken in conjunction with the accompanying drawings in which representative embodiments are described by way of example.

### INCORPORATION BY REFERENCE

All publications, patents, and patent applications mentioned in this specification are herein incorporated by reference to the same extent as if each individual publication, patent, or patent application was specifically and individually indicated to be incorporated by reference. The content of all publications, patents, and patent applications mentioned in this specification are herein incorporated by reference in their entirety for all purposes.

### BRIEF DESCRIPTION OF THE DRAWINGS

FIG. 1 illustrates a schematic view of an imaging system comprising an imaging probe and independent retraction and rotation assemblies, consistent with the present inventive concepts.

FIG. 1A illustrates a schematic view of an imaging system comprising an imaging probe operably attachable to a patient interface module, and an independent pullback module operably attachable to the patient interface module and the imaging probe, consistent with the present inventive concepts.

FIG. 1B illustrates a schematic view of an imaging system comprising an imaging probe operably attachable to a module comprising a first connector for attaching to a rotation motive element and a second connector for attaching to a retraction motive element, consistent with the present inventive concepts.

FIG. 2A illustrates a perspective view of connectors being attached to a patient interface module, consistent with the present inventive concepts.

FIG. 2B illustrates a perspective view of a pullback housing, consistent with the present inventive concepts.

FIG. 3 illustrates a perspective view of connectors being attached to a patient interface module, consistent with the present inventive concepts.

FIGS. 4A and 4B illustrate a schematic view of the distal portion of an imaging probe, and a representation of image data, respectively, consistent with the present inventive concepts.

FIGS. 5A and 5B illustrate representations of image data, consistent with the present inventive concepts.

FIG. 6 illustrates a schematic view of an imaging assembly, consistent with the present inventive concepts.

FIGS. 7A and 7B illustrate sectional anatomic views of an imaging probe within a vessel before and after a pullback procedure, consistent with the present inventive concepts.

FIG. 8 illustrates a schematic view of a portion of an imaging assembly, consistent with the present inventive concepts.

FIGS. 9A and 9B illustrate various views of portions of an imaging probe, consistent with the present inventive concepts.

FIGS. 10A and 10B illustrate sectional views of an unbalanced centrifugal breaking assembly, consistent with the present inventive concepts.

FIGS. 11A and 11B illustrate sectional views of a balanced centrifugal breaking assembly, consistent with the present inventive concepts.

FIGS. 12A and 12B illustrate perspective views of a pullback module, consistent with the present inventive concepts.

### DETAILED DESCRIPTION OF THE DRAWINGS

Reference will now be made in detail to the present embodiments of the technology, examples of which are illustrated in the accompanying drawings. Similar reference numbers may be used to refer to similar components. However, the description is not intended to limit the present disclosure to particular embodiments, and it should be construed as including various modifications, equivalents, and/or alternatives of the embodiments described herein.

It will be understood that the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) when used herein, specify the presence of stated features, integers, steps, operations, elements, and/or components, but do not preclude the presence or addition of one or more other features, integers, steps, operations, elements, components, and/or groups thereof.

It will be further understood that, although the terms first, second, third, etc. may be used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer or section. Thus, a first limitation, element, component, region, layer or section discussed below could be termed a second limitation, element, component, region, layer or section without departing from the teachings of the present application.

It will be further understood that when an element is referred to as being “on”, “attached”, “connected” or “coupled” to another element, it can be directly on or above, or connected or coupled to, the other element, or one or more intervening elements can be present. In contrast, when an element is referred to as being “directly on”, “directly attached”, “directly connected” or “directly coupled” to another element, there are no intervening elements present. Other words used to describe the relationship between elements should be interpreted in a like fashion (e.g. “between” versus “directly between,” “adjacent” versus “directly adjacent,” etc.).

It will be further understood that when a first element is referred to as being “in”, “on” and/or “within” a second element, the first element can be positioned: within an internal space of the second element, within a portion of the second element (e.g. within a wall of the second element); positioned on an external and/or internal surface of the second element; and combinations of one or more of these.

As used herein, the term “proximate”, when used to describe proximity of a first component or location to a second component or location, is to be taken to include one or more locations near to the second component or location, as well as locations in, on and/or within the second component or location. For example, a component positioned proximate an anatomical site (e.g. a target tissue location), shall include components positioned near to the anatomical site, as well as components positioned in, on and/or within the anatomical site.

Spatially relative terms, such as “beneath,” “below,” “lower,” “above,” “upper” and the like may be used to describe an element and/or feature's relationship to another element(s) and/or feature(s) as, for example, illustrated in the figures. It will be further understood that the spatially relative terms are intended to encompass different orientations of the device in use and/or operation in addition to the orientation depicted in the figures. For example, if the device in a figure is turned over, elements described as “below” and/or “beneath” other elements or features would then be oriented “above” the other elements or features. The device can be otherwise oriented (e.g. rotated 90 degrees or at other orientations) and the spatially relative descriptors used herein interpreted accordingly.

The terms “reduce”, “reducing”, “reduction” and the like, where used herein, are to include a reduction in a quantity, including a reduction to zero. Reducing the likelihood of an occurrence shall include prevention of the occurrence. Correspondingly, the terms “prevent”, “preventing”, and “prevention” shall include the acts of “reduce”, “reducing”, and “reduction”, respectively.

The term “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

The term “one or more”, where used herein can mean one, two, three, four, five, six, seven, eight, nine, ten, or more, up to any number.

The terms “and combinations thereof” and “and combinations of these” can each be used herein after a list of items that are to be included singly or collectively. For example, a component, process, and/or other item selected from the group consisting of: A; B; C; and combinations thereof, shall include a set of one or more components that comprise: one, two, three or more of item A; one, two, three or more of item B; and/or one, two, three, or more of item C.

In this specification, unless explicitly stated otherwise, “and” can mean “or”, and “or” can mean “and”. For example, if a feature is described as having A, B, or C, the feature can have A, B, and C, or any combination of A, B, and C. Similarly, if a feature is described as having A, B, and C, the feature can have only one or two of A, B, or C.

As used herein, when a quantifiable parameter is described as having a value “between” a first value X and a second value Y, it shall include the parameter having a value of: at least X, no more than Y, and/or at least X and no more than Y. For example, a length of between 1 and 10 shall include a length of at least 1 (including values greater than 10), a length of less than 10 (including values less than 1), and/or values greater than 1 and less than 10.

The expression “configured (or set) to” used in the present disclosure may be used interchangeably with, for example, the expressions “suitable for”, “having the capacity to”, “designed to”, “adapted to”, “made to” and “capable of” according to a situation. The expression “configured (or set) to” does not mean only “specifically designed to” in hardware. Alternatively, in some situations, the expression “a device configured to” may mean that the device “can” operate together with another device or component.

As used herein, the terms “about” or “approximately” shall refer to ±30%.

As used herein, the term “threshold” refers to a maximum level, a minimum level, and/or range of values correlating to a desired or undesired state. In some embodiments, a system parameter is maintained above a minimum threshold, below a maximum threshold, within a threshold range of values, and/or outside a threshold range of values, such as to cause a desired effect (e.g. efficacious therapy) and/or to prevent or otherwise reduce (hereinafter “prevent”) an undesired event (e.g. a device and/or clinical adverse event). In some embodiments, a system parameter is maintained above a first threshold (e.g. above a first temperature threshold to cause a desired therapeutic effect to tissue) and below a second threshold (e.g. below a second temperature threshold to prevent undesired tissue damage). In some embodiments, a threshold value is determined to include a safety margin, such as to account for patient variability, system variability, tolerances, and the like. As used herein, “exceeding a threshold” relates to a parameter going above a maximum threshold, below a minimum threshold, within a range of threshold values and/or outside of a range of threshold values.

As described herein, “room pressure” shall mean pressure of the environment surrounding the systems and devices of the present inventive concepts. Positive pressure includes pressure above room pressure or simply a pressure that is greater than another pressure, such as a positive differential pressure across a fluid pathway component such as a valve. Negative pressure includes pressure below room pressure or a pressure that is less than another pressure, such as a negative differential pressure across a fluid component pathway such as a valve. Negative pressure can include a vacuum but does not imply a pressure below room pressure. As used herein, the term “vacuum” can be used to refer to a full or partial vacuum, or any negative pressure as described herein.

The term “diameter” where used herein to describe a non-circular geometry is to be taken as the diameter of a hypothetical circle approximating the geometry being described. For example, when describing a cross section, such as the cross section of a component, the term “diameter” shall be taken to represent the diameter of a hypothetical circle with the same cross sectional area as the cross section of the component being described.

The terms “major axis” and “minor axis” of a component where used herein are the length and diameter, respectively, of the smallest volume hypothetical cylinder which can completely surround the component.

As used herein, the term “functional element” is to be taken to include one or more elements constructed and arranged to perform a function. A functional element can comprise a sensor and/or a transducer. In some embodiments, a functional element is configured to deliver energy and/or otherwise treat tissue (e.g. a functional element configured as a treatment element). Alternatively or additionally, a functional element (e.g. a functional element comprising a sensor) can be configured to record one or more parameters, such as a patient physiologic parameter; a patient anatomical parameter (e.g. a tissue geometry parameter); a patient environment parameter; and/or a system parameter. In some embodiments, a sensor or other functional element is configured to perform a diagnostic function (e.g. to gather data used to perform a diagnosis). In some embodiments, a functional element is configured to perform a therapeutic function (e.g. to deliver therapeutic energy and/or a therapeutic agent). In some embodiments, a functional element comprises one or more elements constructed and arranged to perform a function selected from the group consisting of: deliver energy; extract energy (e.g. to cool a component); deliver a drug or other agent; manipulate a system component or patient tissue; record or otherwise sense a parameter such as a patient physiologic parameter or a system parameter; and combinations of one or more of these. A functional element can comprise a fluid and/or a fluid delivery system. A functional element can comprise a reservoir, such as an expandable balloon or other fluid-maintaining reservoir. A “functional assembly” can comprise an assembly constructed and arranged to perform a function, such as a diagnostic and/or therapeutic function. A functional assembly can comprise an expandable assembly. A functional assembly can comprise one or more functional elements.

The term “transducer” where used herein is to be taken to include any component or combination of components that receives energy or any input, and produces an output. For example, a transducer can include an electrode that receives electrical energy, and distributes the electrical energy to tissue (e.g. based on the size of the electrode). In some configurations, a transducer converts an electrical signal into any output, such as: light (e.g. a transducer comprising a light emitting diode or light bulb), sound (e.g. a transducer comprising a piezo crystal configured to deliver ultrasound energy); pressure (e.g. an applied pressure or force); heat energy; cryogenic energy; chemical energy; mechanical energy (e.g. a transducer comprising a motor or a solenoid); magnetic energy; and/or a different electrical signal (e.g. different than the input signal to the transducer). Alternatively or additionally, a transducer can convert a physical quantity (e.g. variations in a physical quantity) into an electrical signal. A transducer can include any component that delivers energy and/or an agent to tissue, such as a transducer configured to deliver one or more of: electrical energy to tissue (e.g. a transducer comprising one or more electrodes); light energy to tissue (e.g. a transducer comprising a laser, light emitting diode and/or optical component such as a lens or prism); mechanical energy to tissue (e.g. a transducer comprising a tissue manipulating element); sound energy to tissue (e.g. a transducer comprising a piezo crystal); chemical energy; electromagnetic energy; magnetic energy; and combinations of one or more of these.

As used herein, the term “fluid” can refer to a liquid, gas, gel, or any flowable material, such as a material which can be propelled through a lumen and/or opening.

As used herein, the term “material” can refer to a single material, or a combination of two, three, four, or more materials.

As used herein, the term “lesion” comprises a segment of a blood vessel (e.g. an artery) that is in an undesired state. As used herein, lesion shall include a narrowing of a blood vessel (e.g. a stenosis), and/or a segment of a blood vessel, with or without narrowing, that includes a buildup of calcium, lipids, cholesterol, and/or other plaque.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination. For example, it will be appreciated that all features set out in any of the claims (whether independent or dependent) can be combined in any given way.

It is to be understood that at least some of the figures and descriptions of the invention have been simplified to focus on elements that are relevant for a clear understanding of the invention, while eliminating, for purposes of clarity, other elements that those of ordinary skill in the art will appreciate may also comprise a portion of the invention. However, because such elements are well known in the art, and because they do not necessarily facilitate a better understanding of the invention, a description of such elements is not provided herein.

Terms defined in the present disclosure are only used for describing specific embodiments of the present disclosure and are not intended to limit the scope of the present disclosure. Terms provided in singular forms are intended to include plural forms as well, unless the context clearly indicates otherwise. All of the terms used herein, including technical or scientific terms, have the same meanings as those generally understood by an ordinary person skilled in the related art, unless otherwise defined herein. Terms defined in a generally used dictionary should be interpreted as having meanings that are the same as or similar to the contextual meanings of the relevant technology and should not be interpreted as having ideal or exaggerated meanings, unless expressly so defined herein. In some cases, terms defined in the present disclosure should not be interpreted to exclude the embodiments of the present disclosure.

Provided herein are imaging systems for a patient comprising an imaging probe and an imaging assembly. The imaging probe comprises an elongate shaft, a rotatable optical core, and an optical assembly. The shaft comprises a proximal end, a distal portion, and a lumen extending between the proximal end and the distal portion. The rotatable optical core comprises a proximal end and a distal end, and at least a portion of the rotatable optical core is positioned within the lumen of the elongate shaft. The optical assembly is positioned proximate the distal end of the rotatable optical core, and it is configured to direct light to tissue and collect reflected light from the tissue. The imaging systems can comprise one or more algorithms configured to enhance the performance of the system.

The imaging systems of the present inventive concepts can be used to provide image data representing arteries, veins, and/or other body conduits, and to image one or more devices inserted into those conduits. The imaging system can be used to image tissue and/or other structures outside of the blood vessel and/or other lumen into which the imaging probe is inserted. The imaging systems can provide image data related to healthy tissue, as well as diseased tissue, such as blood vessels including a stenosis, myocardial bridge, and/or other vessel narrowing (“lesion” or “stenosis” herein), and/or blood vessels including an aneurysm. The systems can be configured to provide treatment information, such as when the treatment information is used by an operator (e.g. a clinician of the patient) to plan a treatment and/or to predict a treatment outcome.

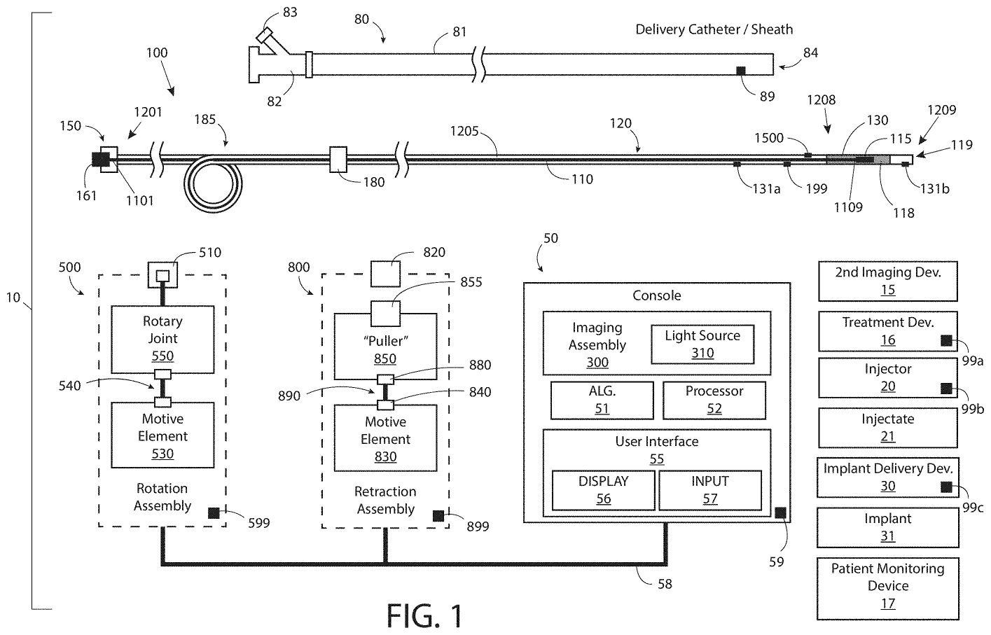

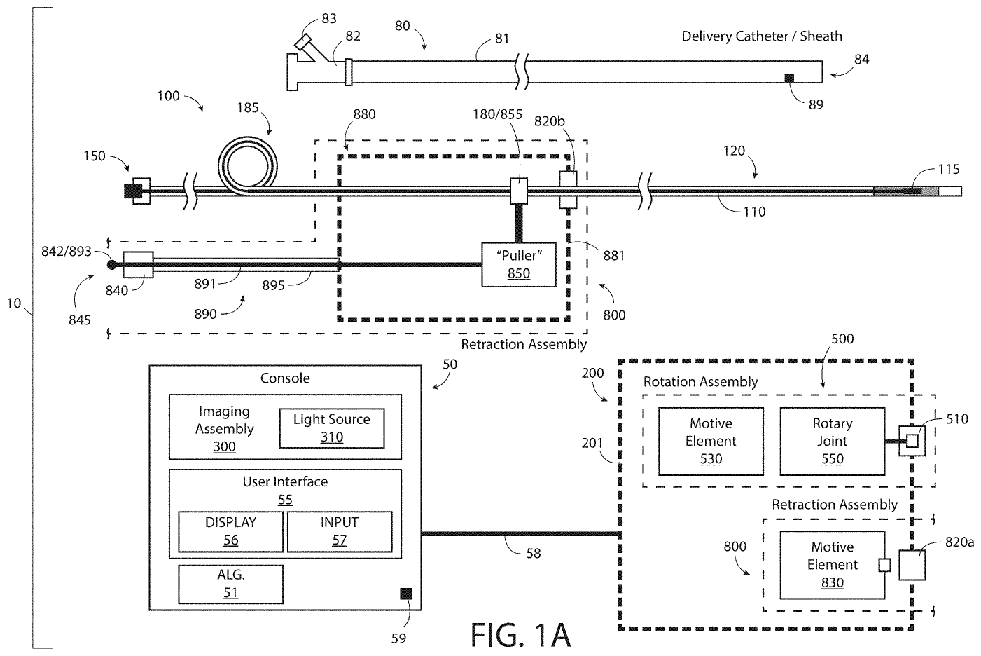

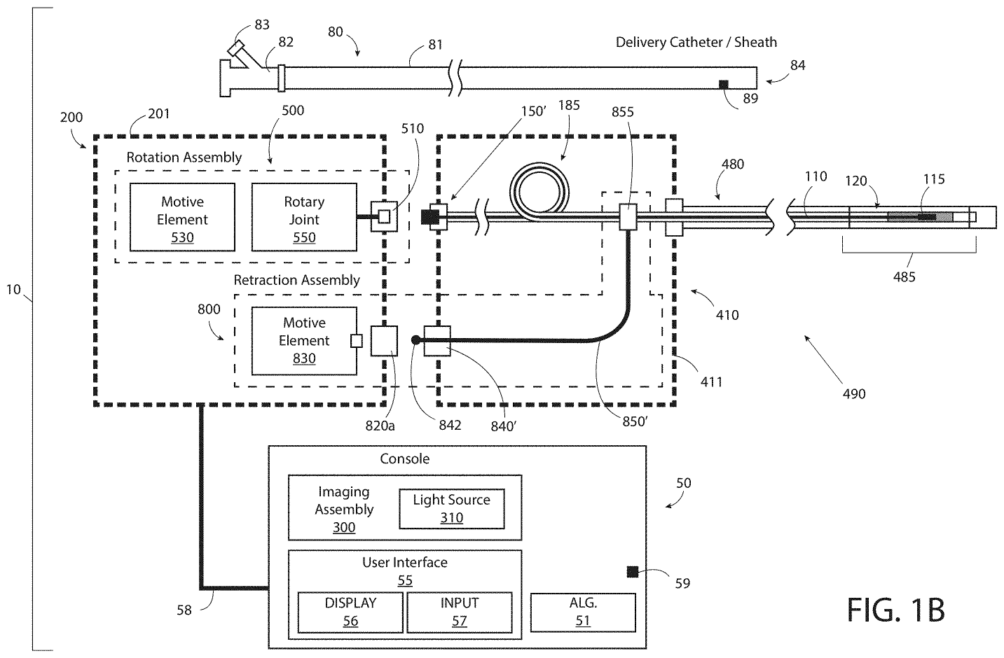

Referring now to FIG. 1, a schematic view of an imaging system comprising an imaging probe and independent retraction and rotation assemblies is illustrated, consistent with the present inventive concepts. Imaging system 10 is constructed and arranged to collect image data and produce one or more images based on the recorded data, such as when imaging system 10 comprises an Optical Coherence Tomography (OCT) imaging system constructed and arranged to collect image data (“image data” or “OCT data” herein) of an imaging location (e.g. a segment of a blood vessel, such as during a pullback procedure). Imaging system 10 comprises a catheter-based probe, imaging probe 100, as well as a rotation assembly 500 and a retraction assembly 800, each of which can operably attach to imaging probe 100. Imaging system 10 can further comprise console 50 which is configured to operably connect to imaging probe 100, such as via rotation assembly 500 and/or retraction assembly 800. Imaging probe 100 can be introduced into a conduit of the patient, such as a blood vessel or other conduit of the patient, using one or more delivery catheters, for example delivery catheter 80 shown. Additionally or alternatively, imaging probe 100 can be introduced through an introducer device, such as an endoscope, arthroscope, balloon dilator, or the like. In some embodiments, imaging probe 100 is configured to be introduced into a conduit selected from the group consisting of: an artery; a vein; an artery within or proximate the heart; a vein within or proximate the heart; an artery within or proximate the brain; a vein within or proximate the brain; a peripheral artery; a peripheral vein; through a natural body orifice into a conduit, such as the esophagus; through a surgically created orifice into a body cavity, such as the abdomen; and combinations of one or more of these. Imaging system 10 can further comprise multiple imaging devices, second imaging device 15 shown. Imaging system 10 can further comprise a device configured to treat the patient, treatment device 16. Imaging system 10 can further comprise one or more devices that are configured to monitor one, two, or more physiologic and/or other parameters of the patient, such as patient monitoring device 17 shown. Imaging system 10 can further comprise a fluid injector, such as injector 20, which can be configured to inject one or more fluids, such as a flushing fluid, an imaging contrast agent (e.g. a radiopaque contrast agent, hereinafter “contrast”) and/or other fluid, such as injectate 21 shown. Imaging system 10 can further comprise an implant, such as implant 31, which can be implanted in the patient via a delivery device, such as an implant delivery device 30 and/or delivery catheter 80.

In some embodiments, imaging probe 100 and/or another component of imaging system 10 can be of similar construction and arrangement to the similar components described in applicants co-pending U.S. patent application Ser. No. 17/668,757 (Docket No. GTY-001-US-CON1), titled “Micro-Optic Probes for Neurology”, filed Feb. 10, 2022, the content of which is incorporated herein by reference in its entirety for all purposes. Imaging probe 100 can be constructed and arranged to collect image data from a patient site, such as an intravascular cardiac site, an intracranial site, or other site accessible via the vasculature of the patient. In some embodiments, imaging system 10 can be of similar construction and arrangement to the similar systems and their methods of use described in applicants co-pending U.S. patent application Ser. No. 17/350,021 (Docket No. GTY-002-US-CON2), titled “Imaging System Includes Imaging Probe and Delivery Devices”, filed Jun. 17, 2021, the content of which is incorporated herein by reference in its entirety for all purposes.

Delivery catheter 80 comprises an elongate shaft, shaft 81, with a lumen 84 therethrough, and a connector 82 positioned on its proximal end. Connector 82 can comprise a Touhy or valved connector, such as a valved connector configured to prevent fluid egress from the associated delivery catheter 80 (with and/or without a separate shaft positioned within the connector 82). Connector 82 can comprise a port 83, such as a port constructed and arranged to allow introduction of fluid into delivery catheter 80 and/or for removing fluids from delivery catheter 80. In some embodiments, a flushing fluid, as described herein, is introduced via one or more ports 83, such as to remove blood or other undesired material from locations proximate optical assembly 115 (e.g. from a location proximal to optical assembly 115 to a location distal to optical assembly 115). Port 83 can be positioned on a side of connector 82 and can include a luer fitting and a cap and/or valve. Shafts 81, connectors 82, and ports 83 can each comprise standard materials and be of similar construction to commercially available introducers, guide catheters, diagnostic catheters, intermediate catheters and microcatheters used in interventional procedures. Delivery catheter 80 can comprise a catheter configured to deliver imaging probe 100 to an intracerebral location, an intracardiac location, and/or another location within a patient.

Imaging system 10 can comprise two or more delivery catheters 80, such as three or more delivery catheters 80. Multiple delivery catheters 80 can comprise at least a vascular introducer, and other delivery catheters 80 that can be inserted into the patient therethrough, after the vascular introducer is positioned through the skin of the patient. Two or more delivery catheters 80 can collectively comprise sets of inner diameters (IDs) and outer diameters (ODs) such that a first delivery catheter 80 slidingly receives a second delivery catheter 80 (e.g. the second delivery catheter OD is less than or equal to the first delivery catheter ID), and the second delivery catheter 80 slidingly receives a third delivery catheter 80 (e.g. the third delivery catheter OD is less than or equal to the second delivery catheter ID), and so on. In these configurations, the first delivery catheter 80 can be advanced to a first anatomical location, the second delivery catheter 80 can be advanced through the first delivery catheter to a second anatomical location distal or otherwise remote (hereinafter “distal”) to the first anatomical location, and so on as appropriate, using sequentially smaller diameter delivery catheters 80. In some embodiments, delivery catheters 80 can be of similar construction and arrangement to the similar components described in applicants co-pending U.S. patent application Ser. No. 17/350,021 (Docket No. GTY-002-US-CON2), titled “Imaging System Includes Imaging Probe and Delivery Devices”, filed Jun. 17, 2021, the content of which is incorporated herein by reference in its entirety for all purposes.

Imaging probe 100 comprises an elongate body, comprising one or more elongate shafts and/or tubes, elongate shaft 120 herein. Shaft 120 comprises a proximal end 1201, distal end 1209, and a lumen 1205 extending therebetween. In some embodiments, lumen 1205 can include multiple coaxial lumens within the one or more elongate shafts 120, such as one or more lumens abutting each other to define a single lumen 1205. In some embodiments, at least a portion of shaft 120 comprises a torque shaft. In some embodiments, a portion of shaft 120 comprises a braided construction. In some embodiments, a portion of shaft 120 comprises a spiral cut tube (e.g. a spiral cut metal tube). In some embodiments, the pitch of the spiral cut can be varied along the length of the cut, such as to vary the stiffness of shaft 120 along the cut. A portion of shaft 120 can comprise a tube constructed of nickel-titanium alloy. Shaft 120 operably surrounds a rotatable optical fiber, optical core 110 (e.g. optical core 110 is positioned within lumen 1205), comprising a proximal end 1101 and a distal end 1109. Optical core 110 can comprise a dispersion shifted optical fiber, such as a depressed cladding dispersion shifted fiber (e.g. a Non-Zero Dispersion Shifted, NZDS, fiber). Shaft 120 further comprises a distal portion 1208, including a transparent window, window 130 (e.g. a window that is relatively transparent to the one or more frequencies of light transmitted through optical core 110). An optical assembly, optical assembly 115, is operably attached to the distal end 1109 of optical core 110. Optical assembly 115 is positioned within window 130 of shaft 120. Optical assembly 115 can comprise a GRIN lens optically coupled to the distal end 1109 of optical core 110. Optical assembly 115 can comprise a construction and arrangement similar to optical assembly 115 as described in applicant's co-pending U.S. patent application Ser. No. 16/764,087 (Docket No. GTY-003-US), titled “Imaging System”, filed May 14, 2020, and applicant's co-pending U.S. patent application Ser. No. 17/276,500 (Docket No. GTY-004-US), titled “Imaging System with Optical Pathway”, filed Mar. 16, 2021, the content of each of which is incorporated herein by reference in its entirety for all purposes. A connector assembly, connector assembly 150, is positioned on the proximal end of shaft 120. Connector assembly 150 operably attaches imaging probe 100 to rotation assembly 500, as described herein. Connector assembly 150 surrounds and operably attaches to an optical connector 161, fixedly attached to the proximal end of optical core 110. A second connector, pullback connector 180, is positioned on shaft 120. Connector 180 can be removably attached and/or adjustably positioned along the length of shaft 120. Connector 180 can be positioned along shaft 120, such as by a clinician, operator or other user of system 10 (“user” or “operator” herein), proximate the proximal end of delivery catheter 80 after imaging probe 100 has been inserted into a patient via delivery catheter 80. Shaft 120 can comprise a portion between connector assembly 150 and the placement location of connector 180 that accommodates slack in shaft 120, a proximal portion of shaft 120 (e.g. a proximal portion of imaging probe 100), service loop 185. In some embodiments, optical core 110 comprises a single length of optical fiber comprising zero splices along its length. In some embodiments, imaging probe 100 comprises a single optical splice, such as a splice being between optical assembly 115 and distal end 1109 of optical core 110 (e.g. when there are zero splices along the length of optical core 110).

In some embodiments, shaft 120 comprises a multi-part construction, such as an assembly of two or more tubes that can be connected in various ways. In some embodiments, one or more tubes of shaft 120 can comprise tubes made of polyethylene terephthalate (PET), such as when a PET tube surrounds the junction between two tubes (e.g. two portions of shaft 120) in an axial arrangement to create a joint between the two tubes. In some embodiments, one or more PET tubes are under tension after assembly (e.g. the tubes are longitudinally stretched when shaft 120 is assembled), such as to prevent or at least reduce the tendency of the PET tube to wrinkle while shaft 120 is advanced through a tortuous path. In some embodiments, one or more portions of shaft 120 include a coating comprising one, two, or more materials and/or surface modifying processes, such as to provide a hydrophilic coating or a lubricious coating. In some embodiments, one or more metal portions of shaft 120 (e.g. nickel-titanium portions) are surrounded by a tube (e.g. a polymer tube), such as to improve the adhesion of a coating to that portion of shaft 120.

Imaging probe 100 can comprise one or more visualizable markers along its length (e.g. along shaft 120), markers 131a-b shown (marker 131 herein). Marker 131 can comprise markers selected from the group consisting of: radiopaque markers; ultrasonically reflective markers; magnetic markers; ferrous material; and combinations of one or more of these. In some embodiments, marker 131 comprises a marker positioned at a location (e.g. a location within and/or at least proximate distal portion 1208) to assist a user of imaging system 10 in performing a pullback procedure (“pullback procedure” or “pullback” herein), such as to cause tip 119 to be positioned at a location distal to the proximal end of an implant after the pullback is completed (e.g. so that imaging probe 100 can be safely advanced through the implant after the pullback).

In some embodiments, imaging probe 100 includes a viscous dampening material, gel 118, positioned within shaft 120 and surrounding optical assembly 115 and a distal portion of optical core 110 (e.g. a gel injected or otherwise installed in a manufacturing process). Gel 118 can comprise a non-Newtonian fluid, for example a shear-thinning fluid. In some embodiments, gel 118 comprises a static viscosity of greater than 500 centipoise, and a shear viscosity that is less than the static viscosity. In these embodiments, the ratio of static viscosity to shear viscosity of gel 118 can be between 1.2:1 and 100:1. In some embodiments, gel 118 is injected from the distal end of window 130 (e.g. in a manufacturing process). In some embodiments, gel 118 comprises a gel which is visualizable under UV light (e.g. when gel 118 includes one or more materials that fluoresce under UV light). In some embodiments, during a manufacturing process when gel 118 is injected into shaft 120 via window 130, shaft 120 is monitored while being illuminated by UV light such that the injection process can be controlled (e.g. injection is stopped when gel 118 sufficiently ingresses into shaft 120). Gel 118 can comprise a gel as described in reference to applicants co-pending U.S. patent application Ser. No. 17/668,757 (Docket No. GTY-001-US-CON1), titled “Micro-Optic Probes for Neurology”, filed Feb. 10, 2022, and applicant's co-pending U.S. patent application Ser. No. 16/764,087 (Docket No. GTY-003-US), titled “Imaging System”, filed May 14, 2020, the content of each of which is incorporated herein by reference in its entirety for all purposes.

Imaging probe 100 can include a distal tip portion, distal tip 119. In some embodiments, distal tip 119 can comprise a spring tip, such as a spring tip configured to improve the “navigability” of imaging probe 100 (e.g. to improve “trackability” and/or “steerability” of imaging probe 100), for example within a tortuous pathway (e.g. within a blood vessel of the brain or heart with a tortuous pathway). In some embodiments, tip 119 comprises a length of between 5 mm and 100 mm (e.g. a spring with a length between 5 mm and 100 mm). In some embodiments, spring tip 119 can comprise a user shapeable spring tip (e.g. at least a portion of spring tip 119 is malleable). Imaging probe 100 can be rotated (e.g. via connector 180) to adjust the direction of a non-linear shaped portion of spring tip 119 (e.g. to adjust the trajectory of spring tip 119 in the vasculature of the patient). Alternatively or additionally, tip 119 can comprise a cap, plug, or other element configured to seal the distal opening of window 130. In some embodiments, tip 119 can comprise a radiopaque marker configured to increase the visibility of imaging probe 100 under an X-ray or fluoroscope. In some embodiments, tip 119 can comprise a relatively short luminal guidewire pathway to allow “rapid exchange” translation of imaging probe 100 over a guidewire of system 10 (guidewire not shown).

In some embodiments, at least the distal portion of imaging probe 100 (e.g. the distal portion of shaft 120 surrounding optical assembly 115) comprises an outer diameter of no more than 0.030″, such as no more than 0.025″, no more than 0.020″, and/or no more than 0.016″.

In some embodiments, imaging probe 100 can be constructed and arranged for use in an intravascular neural procedure (e.g. a procedure in which the blood, vasculature, and other tissue proximate the brain are visualized, and/or devices positioned temporarily or permanently proximate the brain are visualized). An imaging probe 100 configured for use in a neural procedure can comprise an overall length of at least 150 cm, such as a length of approximately 300 cm. Alternatively or additionally, imaging probe 100 can be constructed and arranged for use in an intravascular cardiac procedure (e.g. a procedure in which the blood, vasculature, and other tissue proximate the heart are visualized, and/or devices positioned temporarily or permanently proximate the heart are visualized). An imaging probe 100 configured for use in a cardiovascular procedure can comprise an overall length of at least 120 cm, such as an overall length of approximately 280 cm (e.g. to allow placement of the proximal end of probe 100 outside of the sterile field). In some embodiments, such as for placement outside of the sterile field, imaging probe 100 can comprise a length greater than 220 cm and/or less than 320 cm.

Rotation assembly 500 comprises a connector assembly 510, operably attached to a rotary joint 550. Rotation assembly 500 further comprises a motor or other rotational energy source, motive element 530. Motive element 530 is operably attached to rotary joint 550 via a linkage assembly 540. In some embodiments, linkage assembly 540 comprises one or more gears, belts, pulleys, or other force transfer mechanisms. Motive element 530 can drive (e.g. rotate via linkage assembly 540) rotary joint 550 (and in turn core 110) at speeds of at least 100 rotations per second, such as at least 200 rotations per second, 250 rotations per second, 400 rotations per second, 500 rotations per second, or between 20 rotations per second and 1000 rotations per second. Motive element 530 can comprise a mechanism selected from the group consisting of: a motor; a servo; a stepper motor (e.g. a stepper motor including a gear box); a linear actuator; a hollow core motor; and combinations thereof. In some embodiments, rotation assembly 500 is configured to rotate optical assembly 115 and rotatable core 110 in unison.

Connector assembly 510 operably attaches to connector assembly 150 of imaging probe 100, allowing optical connector 161 to operably engage rotary joint 550. In some embodiments, connector assembly 510 operably engages connector assembly 150. In some embodiments, connector assembly 510 operably engages connector assembly 150 such that rotary joint 550 and optical connector 161 are free to rotate within the engaged assemblies.

Retraction assembly 800 comprises a connector assembly 820, that operably attaches to a reference point, for example connector 82 of delivery catheter 80, such as to establish a reference for retraction assembly 800 relative to the patient. Connector assembly 820 can attach to a reference point such as a patient introduction device, surgical table, and/or another fixed or semi fixed point of reference. A retraction element, puller 850, releasably attaches to connector 180 of imaging probe 100, such as via a carrier 855. Retraction assembly 800 retracts at least a portion of imaging probe 100 (e.g. the portion of imaging probe 100 distal to the attached connector 180), relative to the established reference. In some embodiments, retraction assembly 800 is configured to retract at least a portion of imaging probe 100 (e.g. at least optical assembly 115 and a portion of shaft 120) at a rate of between 5 mm/sec and 100 mm/sec, such as 60 mm/sec. In some embodiments, retraction assembly 800 is configured to retract at least a portion of imaging probe 100 at a rate of at least 60 mm/sec, at least 80 mm/sec, at least 100 mm/sec, and/or at least 150 mm/sec. Additionally or alternatively, the pullback procedure can be performed during a time period of between 0.5 sec and 25 sec, for example approximately 20 sec (e.g. over a distance of 100 mm at 5 mm/sec). Service loop 185 of imaging probe 100 can be positioned between retraction assembly 800 and/or at least connector assembly 820, and rotation assembly 500, such that imaging probe 100 can be retracted relative to the patient while rotation assembly 500 remains stationary (e.g. attached to the surgical table and/or to a portion of console 50).

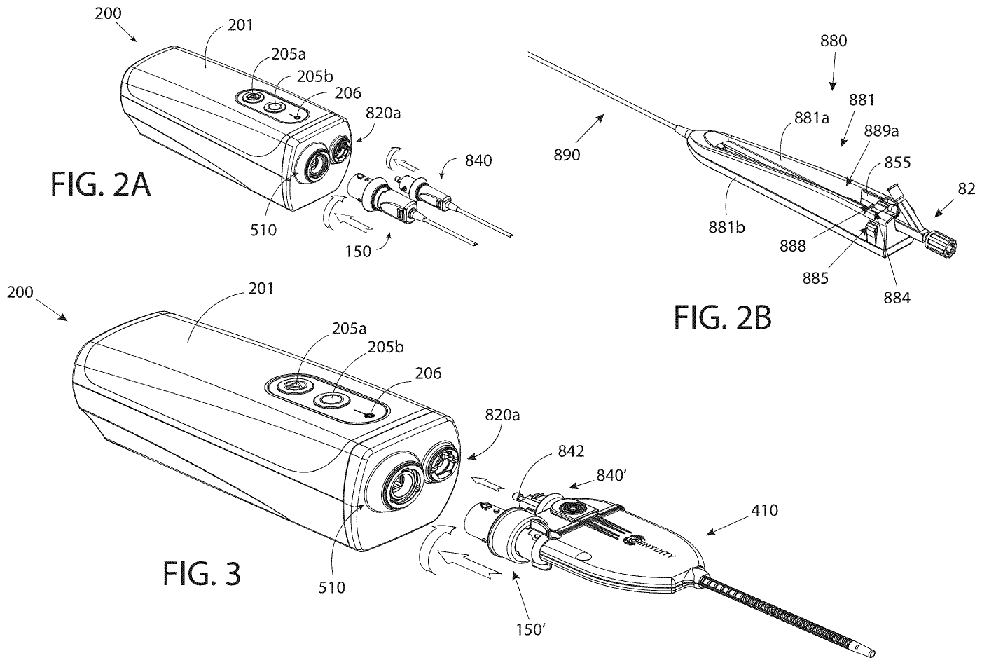

Retraction assembly 800 further comprises a linear drive, motive element 830. In some embodiments, motive element 830 can comprise a linear actuator, a worm drive operably attached to a motor, a pulley system, and/or other linear force transfer mechanisms. Puller 850 can be operably attached to motive element 830 via a linkage assembly 890. In some embodiments, linkage assembly 890 can comprise one or more components of a “pullback assembly”, as described in reference to FIGS. 1A and 2A. Alternatively or additionally, linkage assembly 890 can comprise one or more components of an enclosed pullback connector, as described in reference to FIG. 1B. One or more components of linkage assembly 890 can establish a frame of reference (e.g. a location to be used as an internal pullback reference) between puller 850 and the motive element 830, such that motive element 830 applies a pullback force to puller 850 via linkage assembly 890, and puller 850 retracts relative to the distal portion of linkage assembly 890 (e.g. relative to the distal end of sheath 895 as described in reference to FIG. 1A). In some embodiments, the distal end of linkage assembly 890 and connector assembly 820 are fixed relative to each other, and puller 850 translates linearly between the two in reaction to a force applied from motive element 830.

Console 50 comprises an imaging assembly 300, a user interface 55, processor 52, and one or more algorithms 51. User interface 55 can comprise one or more displays (e.g. a touch screen display), display 56 shown, and one or more user input components (e.g. selectable icons, switches, and/or other user input components), input 57 shown. Processor 52 can include one or more memory storage components, such as one or more memory circuits which store software routines, algorithms (e.g. algorithm 51), and other operating instructions of system 10, as well as data acquired by imaging probe 100, second imaging device 15, and/or another component of system 10. Imaging assembly 300 can be configured to provide light to optical assembly 115 (e.g. via optical core 110) and collect light from optical assembly 115 (e.g. via optical core 110). Imaging assembly 300 can include a light source 310. Light source 310 can comprise one or more light sources, such as one or more light sources configured to provide one or more wavelengths of light to optical assembly 115 via optical core 110. Light source 310 is configured to provide light to optical assembly 115 (via optical core 110) such that image data can be collected comprising cross-sectional, longitudinal and/or volumetric information related to a patient site or implanted device being imaged. Light source 310 can be configured to provide light such that the image data collected includes characteristics of tissue within the patient site being imaged, such as to quantify, qualify or otherwise provide information related to a patient disease or disorder present within the patient site being imaged. Light source 310 can be configured to deliver broadband light and have a center wavelength in the range from 350 nm to 2500 nm, from 800 nm to 1700 nm, from 1280 nm to 1310 nm, or approximately 1300 nm (e.g. light delivered with a sweep range from 1250 nm to 1350 nm). Light source 310 can comprise a sweep rate of at least 50 KHz. In some embodiments, light source 310 comprises a sweep rate of at least 100 KHz, such as at least 200 Khz, 300 KHz, 400 KHz, and/or 500 KHz. These faster sweep rates provide numerous advantages, such as to provide a higher frame rate, where each “frame” is based on, or otherwise represents, the data collected during a single 360° rotation of optical core 110. In addition, the faster sweeper rates are compatible with (e.g. are supportive of) rapid pullback and rotation rates. For example, the higher sweep rate enables the requisite sampling density (e.g. the amount of luminal surface area swept by the rotating beam) to be achieved in a shorter time, advantageous in most situations and especially advantageous when there is relative motion between the probe and the surface/tissue being imaged such as arteries in a beating heart. Light source 310 bandwidth can be selected to achieve a desired resolution, which can vary according to the needs of the intended use of imaging system 10. In some embodiments, bandwidths are about 5% to 15% of the center wavelength, which allows resolutions of between 20 μm and 5 μm. Light source 310 can be configured to deliver light at a power level meeting ANSI Class 1 (“eye safe”) limits, though higher power levels can be employed. In some embodiments, light source 310 delivers light in the 1.3 μm band at a power level of approximately 20 mW. Tissue light scattering is reduced as the center wavelength of delivered light increases, however water absorption increases. Light source 310 can deliver light at a wavelength approximating 1300 nm to balance these two effects. Light source 310 can be configured to deliver shorter wavelength light (e.g. approximately 800 nm light) to traverse patient sites to be imaged including large amounts of fluid. Alternatively or additionally, light source 310 can be configured to deliver longer wavelengths of light (e.g. approximately 1700 nm light), such as to reduce a high level of scattering within a patient site to be imaged. In some embodiments, light source 310 comprises a tunable light source (e.g. light source 310 emits a single wavelength that changes repetitively over time), and/or a broad-band light source. Light source 310 can comprise a single spatial mode light source or a multimode light source (e.g. a multimode light source with spatial filtering).

Light source 310 can comprise a relatively long effective coherence length, such as a coherence length of greater than 10 mm, such as a length of at least 50 mm, at all frequencies within the bandwidth of the light source. This coherence length capability enables longer effective scan ranges to be achieved by system 10, as the light returning from distant objects to be imaged (e.g. tissue) must remain in phase coherence with the returning reference light, in order to produce detectable interference fringes. In the case of a swept-source laser, the instantaneous linewidth is very narrow (i.e. as the laser is sweeping, it is outputting a very narrow frequency band that changes at the sweep rate). Similarly, in the case of a broad-bandwidth source, the detector arrangement must be able to select very narrow linewidths from the spectrum of the source. The coherence length scales inversely with the linewidth. Longer scan ranges enable larger or more distant objects to be imaged (e.g. more distal tissue to be imaged). Current systems have lower coherence length, which correlates to reduced image capture range as well as artifacts (ghosts) that arise from objects outside the effective scan range.

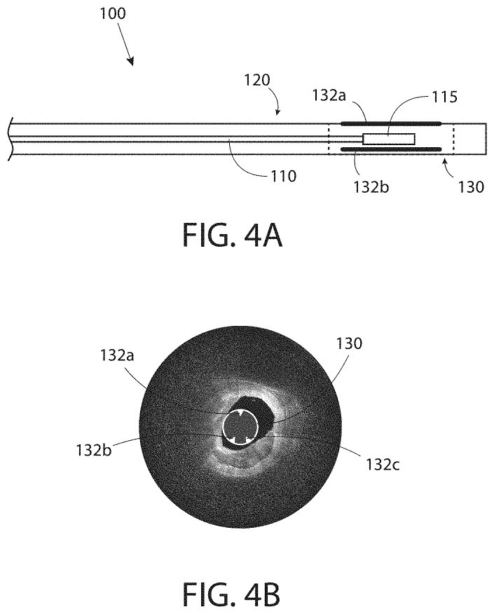

Console 50 can comprise one or more algorithms, such as algorithm 51 shown, which can be configured to adjust (e.g. automatically and/or semi-automatically adjust) one or more operational parameters of imaging system 10, such as an operational parameter of console 50, imaging probe 100 and/or a delivery catheter 80. Console 50 can further comprise a processing assembly, processor 52, configured to execute algorithm 51, and/or perform any type of data processing, such as digital signal processing, described in reference to FIG. 4. Additionally or alternatively, algorithm 51 can be configured to adjust an operational parameter of a separate device, such as injector 20 or implant delivery device 30 described herein. In some embodiments, algorithm 51 is configured to adjust an operational parameter based on one or more sensor signals, such as a sensor signal provided by a sensor-based functional element of the present inventive concepts as described herein. Algorithm 51 can be configured to adjust an operational parameter selected from the group consisting of: a rotational parameter such as rotational velocity of optical core 110 and/or optical assembly 115; a retraction parameter of shaft 120 and/or optical assembly 115 such as retraction velocity, distance, start position, end position and/or retraction initiation timing (e.g. when retraction is initiated); a position parameter such as position of optical assembly 115; a line spacing parameter such as lines per frame; an image display parameter such as a scaling of display size to vessel diameter; an imaging probe 100 configuration parameter; an injectate 21 parameter such as a saline to contrast ratio configured to determine an appropriate index of refraction; a light source 310 parameter such as power delivered and/or frequency of light delivered; and combinations of one or more of these. In some embodiments, algorithm 51 is configured to adjust a retraction parameter such as a parameter triggering the initiation of the pullback, such as a pullback that is initiated based on a parameter selected from the group consisting of: lumen flushing (the lumen proximate optical assembly 115 has been sufficiently cleared of blood or other matter that would interfere with image creation); an indicator signal is received from injector 20 (e.g. a signal indicating sufficient flushing fluid has been delivered); a change in image data collected (e.g. a change in an image is detected, based on the image data collected, that correlates to proper evacuation of blood from around optical assembly 115); and combinations of one or more of these. In some embodiments, algorithm 51 is configured to adjust an imaging system 10 configuration parameter related to imaging probe 100, such as when algorithm 51 identifies (e.g. automatically identifies via an RF or other embedded ID) the attached imaging probe 100 and adjusts an imaging system 10 parameter, such as an optical path length parameter, a dispersion parameter, and/or other parameter as listed above.

In some embodiments, algorithm 51 is configured to trigger the initiation of a pullback based on a time-gated parameter. In some embodiments, a T-wave trigger (e.g. provided by a separate device) can be provided to console 50 to begin pullback when the low-motion portion of the heart cycle is detected. As an alternative to a T-wave trigger, or in addition to it, motion patterns (e.g. relative motion patterns) can be tracked (e.g. using angiography) between one or more portions (e.g. components or other features) of probe 100 and relatively stable (e.g. non-moving) portions of the patient's anatomy (e.g. ribs, sternum and/or spinal column).

When a console 50 of system 10 is first installed at a clinical site (e.g. a catheter lab), a simple calibration routine can be used to establish the latency between the angiographic system and system 10. Essentially, a probe 100 is provided, an angiographic system at the clinical site is engaged and an angiographic image feed is provided to console 50 (e.g. using any standard video connection, analog or digital). Angiographic system-provided video frames are registered according to a clock of console 50, which is used as a reference time frame. A pullback (e.g. in a patient or in a non-patient simulation mode) of probe 100 is initiated (also coordinated by the console 50 clock) and captured by angiography. A trained user or technician reviews the angiographic image frames and designates the first frame in which motion was detected. This process establishes the associated latency according to the console 50 clock. The motion detection can also be automated, for example using a neural network trained to recognize probe 100 movement (e.g. movement of a marker band of probe 100) under angiography.

In some embodiments, a calibration procedure to establish the latency between an angiographic system and system 10, and an imaging procedure performed during relatively low motion of a heart cycle, includes the following steps. In a first step, angiography is initiated once probe 100 has been inserted into the patient and deployed into the target anatomy. In a second step, system 10 analyzes the relative motion between one or more portions of probe 100 (e.g. motion of a marker band or other probe 100 portion which follows the beating heart of the patient) and more stable features in the image, such as images of the sternum or spinal column. Once a cardiac rhythm has been established and the low motion portion identified (typically 5-10 heart cycles are used for this analysis, which can be velocity vector analysis, neural network analysis, and the like), an indicator is provided and a system 10 “metronome” is started. System 10 can reference the output of the metronome, such as at the time that radiopaque flushing material is injected to clear the blood from the target area to be imaged, since the one or more portions of probe 100 (e.g. one or more marker bands) can become radio-invisible during this flushing period. In an alternative embodiment, a non-radiopaque flushing material can be used (e.g. dextran). In a third step, the flushing is started, such as by an operator or in an automated way controlled by system 10. The flushing should continue over several heart cycles, such as 3-5 heart cycles. In a fourth step, clearing of the vessel to be imaged is detected by system 10 analyzing one or more of the images it produces. In a fifth step, at the low motion part of the metronome (e.g. a predicted low motion portion of the heart cycle), and accounting for the latency between system 10 and the angiographic system previously established, a pullback starts. In some embodiments, the pullback will finish in about one-half of a heart cycle or less, such as to remain within the low motion portion of the heart cycle. System 10 can be configured to provide a pullback speed of at least 50 mm/sec, such as at least 100 mm/sec, or 200 mm/sec. In a sixth step, the pullback sequence of images, which include minimal motion artifacts, can be provided to the operator and/or used for CFD calculations, implant (e.g. stent) length measurements, and the like. The use of image capture during low motion, as described herein, avoids errors associated with motion artifacts, notably longitudinal motion artifacts.

In some embodiments, algorithm 51 is configured to perform one, two, or more analyses of the OCT data (e.g. filtering or other image processing analyses) that provide image stabilization (e.g. of displayed OCT data).

Imaging system 10 can comprise one or more interconnect cables, bus 58 shown. Bus 58 can operably connect rotation assembly 500 to console 50, retraction assembly 800 to console 50, and or rotation assembly 500 to retraction assembly 800. Bus 58 can comprise one or more optical transmission fibers, electrical transmission cables, fluid conduits, and combinations of one or more of these. In some embodiments, bus 58 comprises at least an optical transmission fiber that optically couples rotary joint 550 to imaging assembly 300 of console 50. Additionally or alternatively, bus 58 comprises at least power and/or data transmission cables that transfer power and/or motive information to one or more of motive elements 530 and 830.

Second imaging device 15 can comprise an imaging device such as one or more imaging devices selected from the group consisting of: an X-ray; a fluoroscope such as a single plane or biplane fluoroscope; a CT Scanner; an MM; a PET Scanner; an ultrasound imager; and combinations of one or more of these. In some embodiments, second imaging device 15 comprises a device configured to perform rotational angiography.

Treatment device 16 can comprise an occlusion treatment or other treatment device selected from the group consisting of: a balloon catheter constructed and arranged to dilate a stenosis or other narrowing of a blood vessel; a drug eluting balloon; an aspiration catheter; a sonolysis device; an atherectomy device; a thrombus removal device such as a stent retriever device; a Trevo™ stentriever; a Solitaire™ stentriever; a Revive™ stentriever; an Eric™ stentriever; a Lazarus™ stentriever; a stent delivery catheter; a microbraid implant; an embolization system; a WEB™ embolization system; a Luna™ embolization system; a Medina™ embolization system; and combinations of one or more of these. In some embodiments, imaging probe 100 is configured to collect data related to treatment device 16 (e.g. treatment device 16 location, orientation and/or other configuration data), after treatment device 16 has been inserted into the patient.

Patient monitoring device 17 can comprise one or more monitoring devices selected from the group consisting of: an ECG monitor; an EEG monitor; a blood pressure monitor; a blood flow monitor; a respiration monitor; a patient movement monitor; a T-wave trigger monitor; and combinations of these.

Injector 20 can comprise a power injector, syringe pump, peristaltic pump or other fluid delivery device configured to inject a contrast agent, such as radiopaque contrast, and/or other fluids. In some embodiments, injector 20 is configured to deliver contrast and/or other fluid (e.g. contrast, saline and/or Dextran). In some embodiments, injector 20 delivers fluid in a flushing procedure as described herein. In some embodiments, injector 20 delivers contrast or other fluid through a delivery catheter 80 with an ID of between 5 Fr and 9 Fr, a delivery catheter 80 with an ID of between 0.53″ to 0.70″, or a delivery catheter 80 with an ID between 0.0165″ and 0.027″. In some embodiments, contrast or other fluid is delivered through a delivery catheter as small as 4 Fr (e.g. for distal injections). In some embodiments, injector 20 delivers contrast and/or other fluid through the lumen of one or more delivery catheters 80, while one or more smaller delivery catheters 80 also reside within the lumen. In some embodiments, injector 20 is configured to deliver two dissimilar fluids simultaneously and/or sequentially, such as a first fluid delivered from a first reservoir and comprising a first concentration of contrast, and a second fluid from a second reservoir and comprising less or no contrast.

Injectate 21 can comprise fluid selected from the group consisting of: optically transparent material; saline; visualizable material; contrast; Dextran; an ultrasonically reflective material; a magnetic material; and combinations thereof. Injectate 21 can comprise contrast and saline. Injectate 21 can comprise at least 20% contrast. During collection of image data, a flushing procedure can be performed, such as by delivering one or more fluids, injectate 21 (e.g. as propelled by injector 20 or other fluid delivery device), to remove blood or other somewhat opaque material (hereinafter non-transparent material) proximate optical assembly 115 (e.g. to remove non-transparent material between optical assembly 115 and a delivery catheter and/or non-transparent material between optical assembly 115 and a vessel wall), such as to allow light distributed from optical assembly 115 to reach and reflectively return from all tissue and other objects to be imaged. In these flushing embodiments, injectate 21 can comprise an optically transparent material, such as saline. Injectate 21 can comprise one or more visualizable materials, as described herein.

As an alternative or in addition to its use in a flushing procedure, injectate 21 can comprise material configured to be viewed by second imaging device 15, such as when injectate 21 comprises a contrast material configured to be viewed by a second imaging device 15 comprising a fluoroscope or other X-ray device; an ultrasonically reflective material configured to be viewed by a second imaging device 15 comprising an ultrasound imager; and/or a magnetic material configured to be viewed by a second imaging device 15 comprising an Mill.

Implant 31 can comprise an implant (e.g. a temporary or chronic implant) for treating one or more of a vascular occlusion or an aneurysm. In some embodiments, implant 31 comprises one or more implants selected from the group consisting of: a flow diverter; a Pipeline™ flow diverter; a Surpass™ flow diverter; an embolization coil; a stent; a Wingspan™ stent; a covered stent; an aneurysm treatment implant; and combinations of one or more of these.

Implant delivery device 30 can comprise a catheter or other tool used to deliver implant 31, such as when implant 31 comprises a self-expanding or balloon expandable portion. In some embodiments, imaging system 10 comprises imaging probe 100, one or more implants 31 and/or one or more implant delivery devices 30. In some embodiments, imaging probe 100 is configured to collect data related to implant 31 and/or implant delivery device 30 (e.g. implant 31 and/or implant delivery device 30 anatomical location, orientation and/or other configuration data), after implant 31 and/or implant delivery device 30 has been inserted into the patient.

In some embodiments, one or more system components, such as console 50, delivery catheter 80, imaging probe 100, rotation assembly 500, retraction assembly 800, treatment device 16, injector 20, and/or implant delivery device 30, further comprise one or more functional elements (“functional element” herein), such as functional elements 59, 89, 199, 599, 899, 99a, 99b, and/or 99c, respectively, shown. Each functional element can comprise at least two functional elements. Each functional element can comprise one or more elements selected from the group consisting of: sensor; transducer; and combinations thereof. The functional element can comprise a sensor configured to produce a signal. The functional element can comprise a sensor selected from the group consisting of: a physiologic sensor; a pressure sensor; a strain gauge; a position sensor; a GPS sensor; an accelerometer; a temperature sensor; a magnetic sensor; a chemical sensor; a biochemical sensor; a protein sensor; a flow sensor such as an ultrasonic flow sensor; a gas detecting sensor such as an ultrasonic bubble detector; a sound sensor such as an ultrasound sensor; and combinations thereof. The sensor can comprise a physiologic sensor selected from the group consisting of: a pressure sensor such as a blood pressure sensor; a blood gas sensor; a flow sensor such as a blood flow sensor; a temperature sensor such as a blood or other tissue temperature sensor; and combinations thereof. The sensor can comprise a position sensor configured to produce a signal related to a vessel path geometry (e.g. a 2D or 3D vessel path geometry). The sensor can comprise a magnetic sensor. The sensor can comprise a flow sensor. The system can further comprise an algorithm configured to process the signal produced by the sensor-based functional element. Each functional element can comprise one or more transducers. Each functional element can comprise one or more transducers selected from the group consisting of: a heating element such as a heating element configured to deliver sufficient heat to ablate tissue; a cooling element such as a cooling element configured to deliver cryogenic energy to ablate tissue; a sound transducer such as an ultrasound transducer; a vibrational transducer; and combinations thereof.

In some embodiments, imaging probe 100 comprises a fluid propulsion element and/or a fluid pressurization element (“fluid pressurization element” herein), FPE 1500. FPE 1500 can be configured to prevent and/or reduce the presence of bubbles within gel 118 proximate optical assembly 115. FPE 1500 can be fixedly attached to optical core 110, wherein rotation of optical core 110 in turn rotates the fluid propulsion element, such as to generate a pressure increase within gel 118 that is configured to reduce presences of bubbles from locations proximate optical assembly 115. Such one or more fluid pressurization elements FPE 1500 can reduce the likelihood of bubble formation within gel 118, reduce the size of bubbles within gel 118, and/or move any bubbles formed within gel 118 away from a location that would adversely impact the collecting of image data by optical assembly 115 (e.g. move bubbles away from optical assembly 115). In some embodiments, a fluid propulsion element FPE 1500 of imaging probe 100 comprises a similar construction and arrangement to a fluid propulsion element described in applicant's co-pending U.S. patent application Ser. No. 17/600,212 (Docket No. GTY-011-US), titled “Imaging Probe with Fluid Pressurization Element”, filed Sep. 30, 2021, the content of which is incorporated herein by reference in its entirety for all purposes.

In some embodiments, imaging probe 100 comprises an overall length of at least 120 cm, such as at least 160 cm, such as approximately 280 cm. In some embodiments, imaging probe 100 comprises an overall length of no more than 350 cm. In some embodiments, imaging probe 100 comprises a length configured to be inserted into the patient (“insertable length” herein) of at least 90 cm, such as at least 100 cm, such as approximately 145 cm. In some embodiments, imaging probe 100 comprises an insertable length of no more than 250 cm, such as no more than 200 cm. In some embodiments, tip 119 comprises a spring tip with a length of at least 5 mm, such as at least 25 mm, such as approximately 15 mm. In some embodiments, tip 119 comprises a spring tip with a length of no more than 75 mm, such as no more than 30 mm. In some embodiments, a distal portion of shaft 120 (e.g. window 130) comprises an outer diameter of less than 2 fr, such as less than 1.4 fr, such as approximately 1.1 fr. In some embodiments, a distal portion of shaft 120 (e.g. window 130) comprises an outer diameter of at least 0.5 fr, such as at least 0.9 fr. In some embodiments, shaft 120 comprises one or more materials selected from the group consisting of: polyether ether ketone (PEEK); nylon; polyether block amide; nickel-titanium alloy; and combinations of these.

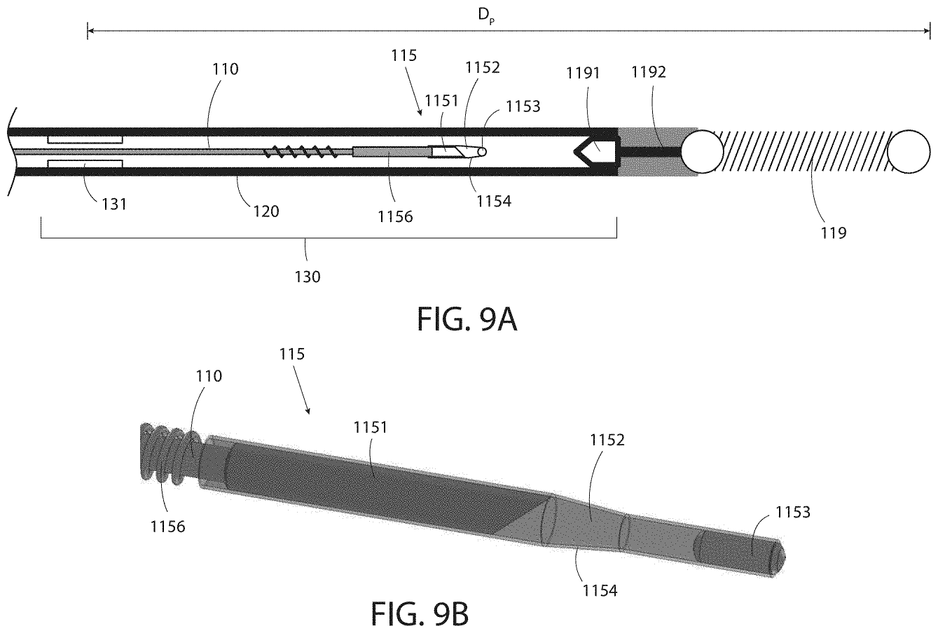

In some embodiments, at least a portion of imaging probe 100 (e.g. the most flexible portion) comprises a minimum radius of curvature of less than 5 mm, such as less than 4 mm, such as less than 3 mm, such as less than 2 mm, such as approximately 1 mm. In some embodiments optical core 110 comprises an optical fiber with a diameter of less than 120 μm, such as less than 100 μm, such as less than 80 μm, such as less than 60 μm, such as approximately 40 μm. In some embodiments, optical core 110 comprises a numerical aperture of one or more of 0.11, 0.14, 0.16, 0.17, 0.18, 0.20, and/or 0.25. In some embodiments, optical assembly 115 comprises a lens selected from the group consisting of: a GRIN lens; a molded lens; a shaped lens, such as a melted and polished lens; a lens comprising an axicon structure, (e.g. an axicon nano-structure); and combinations of these. In some embodiments, optical assembly 115 comprises a lens with an outer diameter of less than 200 μm, such as less than 170 μm, such as less than 150 μm, such as less than 100 μm, such as approximately 80 μm. In some embodiments optical assembly 115 comprises a lens with a length of less than 3 mm, such as less than 1.5 mm. In some embodiments, optical assembly 115 comprises a lens with a length of at least 0.5 mm, such as at least 1 mm. In some embodiments, optical assembly 115 comprises a lens with a focal length of at least 0.5 mm and/or no more than 5.0 mm, such as at least 1.0 mm and/or no more than 3.0 mm, such as a focal length of approximately 0.5 mm. In some embodiments, optical assembly 115 can comprise longer focal lengths, such as to view structures outside of the blood vessel in which optical assembly 115 is inserted, such as is described herebelow in reference to FIG. 9. In some embodiments, optical assembly 115 has a working distance (also termed depth of field, confocal distance, or Rayleigh Range) of up to 1 mm, such as up to 5 mm, such as up to 10 mm, such as a working distance of at least 1 mm and/or no more than 5 mm. In some embodiments, optical assembly 115 comprises an outer diameter of at least 80 μm and/or no more than 200 μm, such as at least 150 μm and/or no more than 170 μm, such as an outer diameter of approximately 150 μm. In some embodiments, system 10 (e.g. retraction assembly 800) is configured to perform a pullback of probe 100 at a speed of at least 10 mm/sec and/or no more than 300 mm/sec, such as at least 50 mm/sec and/or no more than 200 mm/sec, such as a pullback speed of approximately 100 mm/sec. In some embodiments, system 10 (e.g. retraction assembly 800) is configured to perform a pullback for a distance of at least 25 mm and/or no more than 200 mm, such as at least 25 mm and/or no more than 150 mm, such as a distance of approximately 50 mm. In some embodiments, system 10 (e.g. retraction assembly 800) is configured to perform a pullback over a time period of at least 0.2 seconds and/or no more than 5.0 seconds, such as at least 0.5 seconds and/or no more than 2.0 seconds, such as a time period of approximately 1.0 second. In some embodiments, system 10 (e.g. rotation assembly 500) is configured to rotate optical core 110 at an angular velocity of at least 20 rotations per second and/or no more than 1000 rotations per second, such as at least 100 rotations per second and/or no more than 500 rotations per second, such as an angular velocity of approximately 250 rotations per second. In some embodiments, delivery catheter 80 comprises an inner diameter of at least 0.016″ and/or no more than 0.050″, such as at least 0.016″ and/or no more than 0.027″, such as an inner diameter of approximately 0.021″. In some embodiments, light source 310 comprises a sweep rate of at least 20 kHz and/or no more than 2000 kHz, such as at least 50 kHz and/or no more than 500 kHz, such as a sweep rate of approximately 200 kHz. In some embodiments, light source 310 comprises a sweep bandwidth of at least 30 nm and/or no more than 250 nm, such as at least 50 nm and/or no more than 150 nm, such as a sweep bandwidth of approximately 100 nm. In some embodiments, light source 310 comprises a center wavelength of at least 800 nm and/or no more than 1800 nm, such as at least 1200 nm and/or no more than 1350 nm, such as a center wavelength of approximately 1300 nm. In some embodiments, light source 310 comprises an optical power of at least 5 mW and/or no more than 500 mW, such as at least 10 mW and/or no more than 50 mW, such as an optical power of approximately 20 mW.

Referring now to FIG. 1A, a schematic view of an imaging system is illustrated, the system comprising an imaging probe operably attachable to a patient interface module, and an independent pullback module operably attachable to the patient interface module and the imaging probe, consistent with the present inventive concepts. Imaging system 10 can comprise a patient interface module 200. Patient interface module 200 comprises a housing, housing 201, surrounding at least a portion of rotation assembly 500, and at least a portion of retraction assembly 800. Imaging system 10 can further comprise a second, discrete component, pullback module 880. Pullback module 880 comprises a housing, housing 881, surrounding at least a portion of retraction assembly 800. Pullback module 880 and patient interface module 200 can be operably attached to each other via a connector assembly, linkage assembly 890 described herein. Pullback module 880 and patient interface module 200 can be constructed and arranged (via each having a separate housing) to enable positioning at different locations (e.g. linkage assembly 890 connecting modules 880 and 200 can comprise a length of at least 15 cm such that the two remote locations can be at least 15 cm apart), for example patient interface module 200 can be positioned on or near a surgical bed rail, and pullback module 880 can be positioned near a vascular access site of the patient (e.g. within 30 cm of the vascular access site thru which imaging probe 100 enters the patient). Linkage assembly 890 can comprise a linkage 891 slidingly received within sheath 895. Linkage 891 is operably attached to puller 850, and the proximal end 893 of linkage 891 can comprise a connection point, 842. Components shown in FIG. 1A can be of similar construction and arrangement to like components described in reference to FIG. 1, and as described elsewhere herein.

Pullback module 880 can comprise a connector assembly 820b that operably attaches to connector 82 of delivery catheter 80, such as described in reference to FIG. 2B. Connector assembly 845 can comprise a connector 840 that operably attaches to a connector assembly 820a of patient interface module 200, as described in reference to FIG. 2A.

Referring now to FIG. 1B, a schematic view of an imaging system is illustrated, the system comprising an imaging probe operably attachable to a module comprising a first connector for attaching to a rotation motive element and a second connector for attaching to a retraction motive element, consistent with the present inventive concepts. Imaging system 10 can comprise a patient interface module 200 as described herein. Imaging system 10 can further comprise a connector module, module 410. Module 410 comprises a housing, housing 411, surrounding at least a portion of retraction assembly 800, service loop 185 of imaging probe 100, connector assembly 150′, and connector 840′. Module 410 can be configured to operably attach both imaging probe 100 and a linkage, puller 850′, to patient interface module 200. Components shown in FIG. 1B can be of similar construction and arrangement to like components described in reference to FIG. 1, and as described elsewhere herein. Module 410 can be operably attached to a delivery catheter 480. Delivery catheter 480 can be of similar construction and arrangement to delivery catheter 80 described in reference to FIG. 1. Delivery catheter 480 can comprise at least a portion that is optically transparent, window 485. Window 485 can be positioned at or near a distal portion of delivery catheter 480. Window 485 can comprise a material transparent to imaging modalities utilized by imaging probe 100, such that imaging probe 100 can image through window 485, for example when optical assembly 115 is retracted within window 485. In some embodiments, module 410, delivery catheter 480, and imaging probe 100 collectively form catheter assembly 490.

Referring now to FIG. 2A, a perspective view of connectors being attached to a patient interface is illustrated, consistent with the present inventive concepts. Patient interface module 200 is configured to provide rotation to a rotatable optical core of an imaging probe, and to provide a motive force to translate at least a portion of the imaging probe, such as is described herein. Patient interface module 200 comprises rotation assembly 500, and at least a portion of retraction assembly 800. A housing 201 surrounds patient interface module 200. Patient interface module 200 can comprise one or more user interface elements, such as one or more inputs, buttons 205a,b, and one or more outputs, indicator 206 shown. Patient interface module 200 comprises a first physical connector assembly, connector assembly 510, for operably connecting to connector assembly 150 as described herein. Patient interface module 200 can further comprise a second physical connector assembly, connector assembly 820a, for operably connecting to connector 840 also as described herein. Connector assembly 150 and connector 840 can each comprise bayonet type connectors, constructed and arranged to be at least partially inserted into connector assemblies 510 and 820a, respectively. Connector assembly 150 and connector 840 can be subsequently rotated (e.g. an approximately 45° rotation) to lock their connections with connector assemblies 510 and 820a, respectively, as described herein. Connector assembly 150 and/or connector 840 can comprise numerous forms of connectors, such as a bayonet or other locking connectors.

Referring now to FIG. 2B, a perspective view of a pullback assembly is illustrated, consistent with the present inventive concepts. Pullback module 880 can be operably attached to a portion of an imaging probe 100 of the present inventive concepts, and provide a retraction force to the probe, pulling at least a portion of the probe proximally relative to a patient (e.g. relative to a patient introduction device), as described herein. Pullback module 880 can comprise a construction and arrangement similar to pullback module 880 as described in applicant's co-pending U.S. patent application Ser. No. 16/764,087 (Docket No. GTY-003-US), titled “Imaging System”, filed May 14, 2020, the content of which is incorporated herein by reference in its entirety. Pullback module 880 can be operably attached to the distal end of a linkage 891 (not shown). Linkage assembly 890 can be slidingly received through pullback module 880. Sheath 895 can be fixedly attached to the proximal end of module 880. Linkage 891 is slidingly received along the length of module 880 and is operably attached at its distal end to puller 850.

Pullback module 880 can comprise a two-part housing 881, including a top housing 881a and bottom housing 881b. Module 880 can contain a translating cart, puller 850 (not shown, but positioned below carrier 855, and as described herein). Puller 850 can be designed to translate within module 880. Module 880 can comprise a biasing element, spring 852 (not shown). Spring 852 can provide a biasing force to puller 850, such as to bias puller 850 distally.

Top housing 881a can comprise a first cavity, retention port 884 and a second cavity, trench 889. Retention port 884 and trench 889 can be separated by a projection, retention wall 888. Physical connector assembly 820b can comprise a retention port 884 of housing 881a, including wall 888, and a retention mechanism, clip 885. Clip 885 can be configured to releasably engage the proximal end of a delivery catheter such as sheath connector 82 of delivery catheter 80, such as when connector 82 comprises a Tuohy Borst connector. Physical connector assembly 820b can further comprise a biasing element, spring 886 (not shown). Spring 886 can provide a biasing force to maintain clip 885 in an engaged position about connector 82.

Pullback module 880 can further comprise a carrier 855. Carrier 855 can operably attach to puller 850, such as through a slot 889a in housing 881a. Carrier 855 can translate within trench 889 in response to puller 850, which translates in response to linkage 891. Carrier 855 can operably attach to a portion of imaging probe 100, such as to a pullback connector 180. Pullback connector 180 can comprise a “torquer”, or other device affixed to shaft 120 of imaging probe 100. Sheath 895 of linkage assembly 890 can provide a frame of reference between connector 840 and pullback module 880, such that when the proximal end of linkage 891 is retracted relative to connector 840, the distal end of linkage 891 is retracted towards sheath 895 (i.e. towards the proximal end of pullback module 880). This relative motion transfers motive force applied at connector 840 (e.g. via motive element 830, as described herein), to puller 850. Puller 850 subsequently transfers the motive force to imaging probe 100, and imaging probe 100 is retracted relative to the patient.

In operation, imaging probe 100 can be manually (e.g. by a clinician of the patient) advanced through the vasculature of the patient. Pullback module 880 can be attached to the patient (e.g. to delivery catheter 80 via connector 82), and connector 180 can be operably connected to imaging probe 100 and positioned proximate delivery catheter 80 (e.g. a torquer connector 180 can be tightened to imaging probe 100 proximate delivery catheter 80). Connector 180 (not shown) can be operably positioned within carrier 855, and a motive force can be applied to the distal end of linkage 891. Carrier 855 retracts within trench 889, retracting imaging probe 100 relative to the patient. After retraction, connector 180 can be removed from carrier 855 (e.g. lifted out of), and carrier 855 and imaging probe 100 can be re-advanced independently. For example, carrier 855 can re-advance via the bias of spring 852, as the proximal end of linkage 891 is allowed to advance, and imaging probe 100 can be re-advanced manually by an operator of system 10. Subsequent retractions can be performed by repositioning connector 180 in carrier 855 after both have been re-advanced. Carrier 855 can comprise a capturing portion, such as a “cup-like” geometry, a hook, or other capture-enabling portion, such that carrier 855 can only impart a retraction force on connector 180. In this configuration, if carrier 855 were to translate distally, connector 180 would automatically disengage from carrier 855 (e.g. connector 180 would fall out of the cup portion of carrier 855).

Carrier 855 can comprise a two-piece assembly which enables micro-adjustability of the carrier 855 to accommodate variations in the positioning of pullback connector 180 relative to delivery catheter 80. The adjustability of the two-piece assembly is laterally constrained but is allowed to be adjusted axially. Carrier 855 can comprise one or more user graspable projections, and one or more toothed features on a first portion of the two-piece assembly that engage with notched features on a second portion of the two-piece assembly, thereby locking the components together during use. By depressing the projections, carrier 855 can be adjusted and locked into a new position.

Referring now to FIG. 3, a perspective view of connectors being attached to a patient interface module is illustrated, consistent with the present inventive concepts. Patient interface module 200 can be of similar construction and arrangement to patient interface module 200 as described in reference to FIG. 2A. Patient interface module 200 comprises a first physical connector assembly, connector assembly 510, for operably connecting to connector assembly 150′. Patient interface module 200 can further comprise a second physical connector assembly, connector assembly 820a, for operably connecting to connector 840′. Connector assembly 150′ and connector 840′ can each comprise bayonet type connectors, constructed and arranged to be at least partially inserted into connector assemblies 510 and 820a, respectively.

As described herein, system 10 can be constructed and arranged to provide improved imaging of a patient's anatomy (e.g. of one or more blood vessels of the patient) as well as improved imaging of implants, catheters, and/or other devices positioned in the patient (e.g. positioned in a blood vessel of the patient). In some embodiments, system 10 is configured to provide information that is used (e.g. by a clinician) to perform a treatment (e.g. an intervention), wherein the information is based on, at least, optical coherence tomography data. For example, OCT and other data gathered by system 10, can be used to plan a treatment and/or predict a treatment outcome (e.g. the planning and/or predicting performed by system 10, an operator of system 10, or a combination of the two), such as to impact a treatment to be delivered to the patient (“OCT-guided treatment” and/or “OCT-guided therapy” herein).

As described herein, imaging probe 100 can comprise at least one of: size (e.g. diameter and/or length), scan range, flexibility, and/or imaging capability configured to provide the improved imaging. Imaging probe 100 can comprise a size and/or flexibility configured to enable imaging of tight lesions within the vessel. As used herein, a tight lesion can comprise a lesion whose resultant lumen (i.e. the lumen within the lesion) comprises a diameter (e.g. the smallest diameter along the length of the lesion) of less than 2 mm (0.080″). A commercially available OCT catheter positioned to image a lesion with a lumen of this small diameter would effectively block the proximally-applied flush media from propagating to locations distal to the lesion, preventing the use of this commercial device. However, imaging probe 100 can be constructed and arranged to image these tight lesions, for example lesions with a resultant lumen as small as 1.5 mm (0.060″), 1.3 mm (0.053″), 1.1 mm (0.043″), and/or as small as 0.9 mm (0.036″) can be imaged by imaging probe 100. For example, the distal portion of imaging probe 100 can comprise an outer diameter of no more than 2.6 F (0.034″), such as an outer diameter of no more than 1.7 F (0.022″), such as to enable system 10 to be used to image potential vessels (e.g. arteries) to be treated that have a tight lesion, such as when the distal portion of imaging probe 100 is inserted into and through a stenosis, such as in a “pre-treatment” imaging procedure (e.g. a procedure performed prior to intervention or other treatment of the stenosis). As described herein, currently available OCT imaging systems can be too large to provide useful data (e.g. unable to pass thru and/or provide sufficient blood clearing in a tight lesion). Other types of imaging systems, such as angiography, may not provide sufficiently accurate results when imaging tight lesions (e.g. erroneously indicate no treatment is warranted, such as when providing FFR information). In some embodiments, system 10 is used to perform a pre-treatment imaging procedure (e.g. of a tight lesion) to gather data to enable OCT-guided treatment in which the data provided by system 10 (e.g. using images from at least probe 100) is used by an operator (e.g. a clinician) to make decisions about a future treatment to be performed. In these embodiments, system 10 can also be used to image a similar anatomical location, after the treatment has been performed (in a “post-treatment” imaging procedure).

In some embodiments, system 10 is configured to perform a pre-treatment imaging procedure (e.g. of a tight lesion or otherwise) and provide OCT-guided treatment due to the following characteristics of system 10: distal portion of probe 100 (e.g. including optical assembly 115) comprises a diameter of less than 2.6 Fr (0.034″), such as a diameter of no more than 2.0 Fr (0.026″), such as a diameter of no more than 1.7 Fr (0.022″)

In some embodiments, system 10 is configured to perform a pre-treatment imaging procedure (e.g. of a tight lesion or otherwise) and provide OCT-guided treatment due to the following characteristics of system 10: optical assembly 115 is rotated (e.g. via rotation assembly 500) at a rate of more than 180 rotations per second, such as a rate of at least 200, 250, 400, and/or 500 rotations per second.

In some embodiments, system 10 is configured to perform a pre-treatment imaging procedure (e.g. of a tight lesion or otherwise) and provide OCT-guided treatment due to the following characteristics of system 10: scan range of system 10 is at least a radius of 7 mm, such as a radius of at least 11 mm. The long scan range of system 10 provides numerous advantages, such as the ability to image from the imaged vessel into any side branches of that vessel, the ability to image large vessels when optical assembly 115 is eccentrically positioned within the vessel lumen (e.g. proximate a portion of the vessel wall), and/or the ability to image larger vessels in general, such as the left main artery, carotid arteries, and large peripheral arteries.

In some embodiments, system 10 is configured to perform a pre-treatment imaging procedure (e.g. of a tight lesion or otherwise) and provide OCT-guided treatment due to the following characteristics of system 10: pullback distance of more than 7.5 cm, such as a pullback of at least 10 cm, or at least 15 cm. The pullback can be performed at a rate of at least 25 mm/sec, and/or within a time period of no more than 4 seconds (e.g. a complete pullback of at least 7.5 cm, 10 cm, and/or 15 cm in no more than 4 seconds). The operable pullback speed of imaging probe 100 can be determined via a relationship between the rotation rate of optical assembly 115 and the desired frame density (e.g. frames/mm) of the OCT image data, such that the pullback speed comprises the rotation rate divided by the frame density. Imaging probe 100 can comprise a rotation rate of greater than 180 Hz, such as at least 200 Hz or at least 250 Hz. Imaging probe 100 can comprise a frame spacing of no more than 0.2 mm (i.e. a frame density of at least 5 frames/mm). Imaging probe 100 can comprise a laser scan frequency of at least 200 KHz.

In some embodiments, system 10 is configured to perform a pre-treatment imaging procedure (e.g. of a tight lesion or otherwise) and provide OCT-guided treatment due to the following characteristics of system 10: pullback speed (the translation rate of optical assembly 115 during a pullback) of at least 50 mm/sec. In these embodiments, rotation rate of optical assembly 115 can be at least 180 Hz, 200 Hz, and/or 250 Hz. In these embodiments, the frame spacing can be 0.2 mm minimum.

In some embodiments, system 10 is configured to perform a pre-treatment imaging procedure (e.g. of a tight lesion or otherwise) and provide OCT-guided treatment due to the following characteristics of system 10: lines per frame of at least 400, such as at least 800 lines/frame, where a frame comprises approximately 360° of continuous image data (i.e. one full rotation of optical assembly 115 provides one frame of image data). In some embodiments, system 10 is configured to capture frames at a rate sufficient to allow down-sampling of the frames (e.g. down sampling performed prior to analog to digital conversion of the data, and/or other bandwidth-limited data processing).

In some embodiments, system 10 is configured to perform a pre-treatment imaging procedure (e.g. of a tight lesion or otherwise) and provide OCT-guided treatment due to the following characteristics of system 10: scan frequency of at least 50 kHz, such as at least 200 kHz, 350 kHz, and/or 500 kHz. In these embodiments, the lines per frame can be at least 400 lines/frame, or at least 800 lines/frame (e.g. where lines per frame equals the scan frequency divided by the rotation rate of optical assembly 115).

In some embodiments, system 10 comprises a laser scan frequency of no less than 200 kHz, a pullback speed of no less than 60 mm/sec or 100 mm/sec, and/or a rotation rate of no less than 250 Hz. System 10 can be configured to allow imaging of at least 50 mm of a vessel, such as at least 50 mm imaged in no more than 0.5 seconds, with no less than 800 scan lines per rotation, with approximately 400 μm pitch and/or a frame density of at least 2.5 frames/mm, and/or at least 5.0 frames/mm. In some embodiments, system 10 is configured to perform a pullback during a resting portion of the heart cycle to minimize motion artifacts. In some embodiments, system 10 comprises a rotation rate of up to 400 kHz, such as no less than 250 kHz, 300 kHz, or 350 kHz.

In some embodiments, system 10 is configured to perform a pre-treatment imaging procedure (e.g. of a tight lesion or otherwise) and provide OCT-guided treatment due to the following characteristics of system 10: processor 52 is configured to identify (e.g. via algorithm 51) a reflection generated at the splice interface between optical assembly 115 and optical core 110. The optical interface between optical assembly 115 (e.g. optical assembly 115 comprising a GRIN lens) and optical core 110 (e.g. optical core 110 comprising a NZDS fiber) can comprise a relatively large index mismatch, providing a clearly differentiable reflection. This reflection can provide a reference point for the OCT image data collected by system 10. In some embodiments, the interface can be identified by algorithm 51 with or without rotating optical core 110.

Referring now to FIGS. 4A and 4B, a schematic view of the distal portion of an imaging probe, and a representation of image data are illustrated, respectively, consistent with the present inventive concepts. Rotating intravascular imaging catheters (e.g. imaging probe 100) can be affected by mechanical and/or rotational instability (e.g. instability causing non-uniform rotational distortion, or NURD). Mechanical and/or rotational instability can result in misalignment of consecutive frames of image data. In FIG. 4A, the distal portion of imaging probe 100 is shown, including optical core 110, and optical assembly 115. Optical assembly 115 is positioned within window 130 of elongate shaft 120. Window 130 can include one or more imagable portions (e.g. markers), fiducials 132, such as one to ten fiducials, such as the two fiducials 132a,b shown on probe 100 of FIG. 4A, and the three fiducials 132a,b,c shown in the image of FIG. 4B. Algorithm 51 of console 50 can be configured to utilize image information provided by fiducials 132a,b to reduce the negative impact of NURD, as described herebelow.

In some embodiments, fiducials 132 comprise material positioned on and/or within the walls of window 130, such as wire adhered to and/or inserted within the wall of window 130. Alternatively or additionally, fiducials 132 can comprise a modification to a portion of window 130, for example, an imagable pattern that has been laser inscribed into window 130. One or more fiducials 132 can be positioned axially along at least a portion of window 130. Fiducials 132 can be configured to be imaged by imaging assembly 300, such as during a pullback procedure as described herein. For example, a pullback procedure can be performed whereby optical core 110, including optical assembly 115, is rotated within shaft 120 and imaging probe 100 is retracted within a vessel to be imaged, such that optical assembly 115 remains relatively stationary (e.g. longitudinally stationary), relative to window 130 (e.g. window 130 and optical assembly 115 are pulled back in unison). FIG. 4B represents a single frame of image data. As shown in FIG. 4B, the frame includes image data representing the walls of window 130, as well as three fiducials 132a-c. In the illustrated embodiment, fiducial 132a is aligned with the top of window 130, and fiducials 132b,c are centered about the bottom of window 130. Algorithm 51 of console 50 can be configured to rotationally align multiple frames of image data by correlating each frame via the data representing fiducials 132.

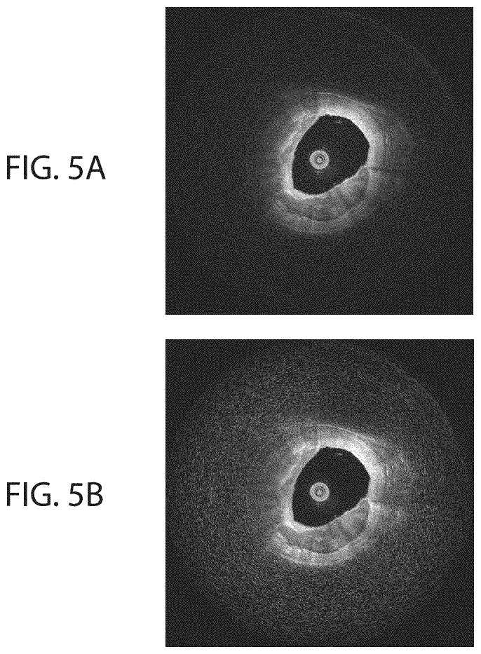

Referring now to FIGS. 5A and 5B, two representations of a frame of image data are illustrated, consistent with the present inventive concepts. In some embodiments, algorithm 51 of console 50 is configured to modify the recorded image data by compensating for one or more insufficiencies of recorded OCT data. For example, one potential insufficiency of OCT image data is that the intensity of the image decays exponentially with depth (e.g. the distance of object being imaged from optical assembly 115). Algorithm 51 can be configured to modify different portions of the image data (e.g. different locations within each frame of the image data) based on one, two, or more characteristics of that portion of data. For example, algorithm 51 can be configured to exponentially increase the intensity (e.g. the brightness) of the image data based on the distance from the center of the frame (e.g. distance of the object being imaged from optical assembly 115). In some embodiments, the image data is compensated based on specific properties of imaging probe 100, such as the focal point or the Raleigh range. Additionally or alternatively, the image data can be compensated based on the physical, optical, and/or other property of the imaging system (e.g. based on the modality of the system), such as the exponential nature of light intensity decay in highly scattering tissue. As an example of image compensation, FIG. 5A illustrates an uncompensated frame of image data, whereas FIG. 5B illustrates the same frame of image data compensated for the decay of light intensity.

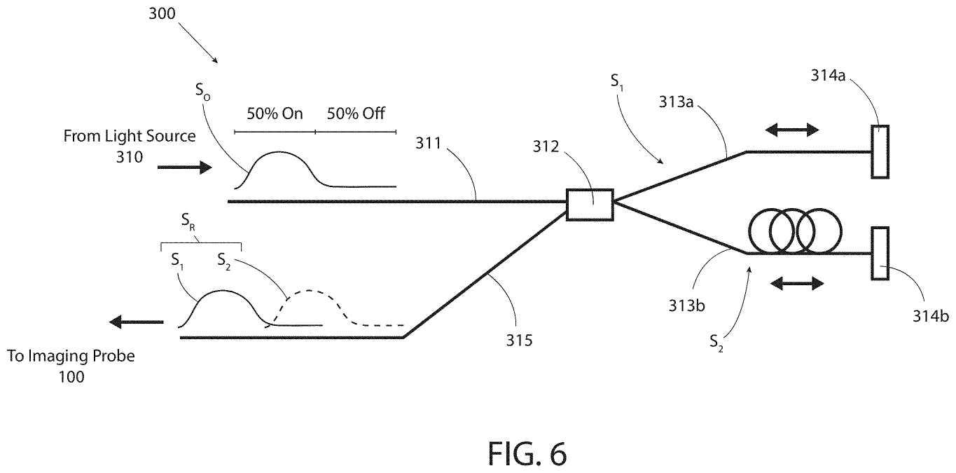

Referring now to FIG. 6, a schematic view of an imaging assembly is illustrated, consistent with the present inventive concepts. FIG. 6 further illustrates a visual representation of a portion of an optical signal carried through the optical elements of imaging assembly 300. Imaging assembly 300 can comprise one, two, or more optical elements constructed and arranged to manipulate the light transmitted from light source 310. Various arrangements of these optical elements provide various benefits to system 10. In some embodiments, physical manipulation of the light transmitted from light source 310 can be used to alter the properties of the light transmitted to imaging probe 100 (e.g. the light transmitted to imaging probe 100 can comprise different properties than the light transmitted from light source 310). As an example, the components of imaging assembly 300 illustrated in FIG. 6 are configured to duplicate and shift the light transmitted from light source 310, such that the light transmitted to imaging probe 100 comprises a duty cycle that is two times the duty cycle of the light source.

In the illustrated embodiment, imaging assembly 300 comprises a first optical fiber, fiber 311, that optically connects light source 310 to an optical splitter, splitter 312. Light source 310 provides a light signal, original light signal S_{O}. Signal S_{O} can comprise a 50% duty cycle, where signal S_{O} comprises minimal (e.g. zero) amplitude approximately half of the time that imaging assembly 300 is active (e.g. imaging assembly 300 is transmitting signal S_{O}). Splitter 312 splits (e.g. equally splits) signal S_{O} into two signals (S₁ and S₂) along two signal paths, such as a first path along a second optical fiber 313a and a second path along third optical fiber 313b. Each of optical fibers 313a,b terminate at a mirror, mirrors 314a,b, respectively, configured to reflect signals S₁ and S₂ back to splitter 312. Optical fiber 313b comprises a length greater than the length of optical fiber 313a, whereby the difference in length is selected such that signal S₂ is delayed by a time equal to one-half of the period of signal S_{O} (e.g. signal S₂ is delayed one-half period behind signal S₁ when signals S₁ and S₂ return to splitter 312). Splitter 312 can be configured to recombine the two signals reflected by mirrors 314a,b, and to transmit this recombined signal along a fourth optical fiber, fiber 315, to imaging probe 100. The recombined signal, signal S_{R}, includes signal S₁ and signal S₂, whereby signal S₂ comprises signal S₁ offset by one-half of the period of signal S_{O} and therefore falls into the time period where signal S₁ is inactive. In this manner, signal S_{R} comprises a greater duty cycle than S_{O} (e.g. S_{R} comprises a 100% duty cycle signal).

This physical duplicating of the active portion of S_{O} can provide various benefits: the duplication allows doubling the sweep speed of the laser (for example from 200 kHz to 400 kHz), or reduction of the sweep speed by 50% (from 200 kHz to 100 kHz), while still collecting the same amount of information (e.g. the same as the amount of information captured at 200 kHz). In the reduction configuration, it is possible to maintain the same amount the information (captured at 200 kHz), but at the same time, slow down the laser sweep by 50%, and allow the use of a slower k-clock which can allow reduced complexity electronics (e.g. reduced cost of detector, digitizer, and/or other electronic components).

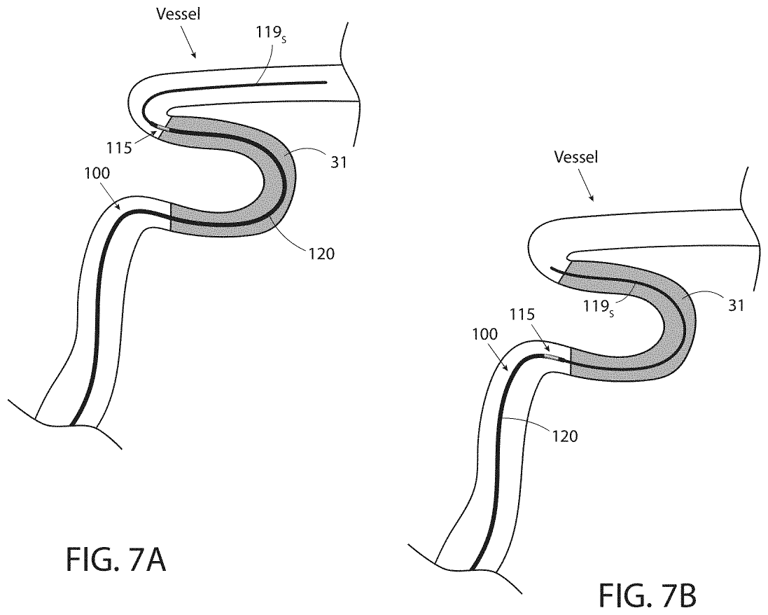

Referring now to FIGS. 7A and 7B, sectional anatomic views of an imaging probe within a vessel prior to and after a pullback procedure are illustrated, respectively, consistent with the present inventive concepts. FIG. 7A illustrates imaging probe 100 positioned within a vessel prior to a pullback imaging procedure. In FIG. 7A, optical assembly 115 is positioned in the vessel at the distal end of implant 31, which has been previously implanted into the vessel (e.g. in the same clinical procedure as FIG. 7A, or in a previous clinical procedure performed days, weeks, or months prior). Imaging probe 100 comprises a distal spring tip, spring tip 119s, that extends distally from the distal end of shaft 120. FIG. 7B illustrates imaging probe 100 positioned within the same vessel, after the pullback procedure has been completed. Optical assembly 115 is now positioned proximal to the proximal end of implant 31, indicating that the pullback imaging procedure captured image data along the length of the vessel between the location of optical assembly 115 in FIGS. 7A and 7B. In FIG. 7B, spring tip 119s extends beyond the pullback starting location of optical assembly 115 (e.g. the initial location of optical assembly 115 at the start of the pullback procedure as shown in FIG. 7A). The position of the distal end of spring tip 119s of FIG. 7B, provides for simplified (e.g. safe) advancement of imaging probe 100 to the location shown in FIG. 7A (e.g. to again position optical assembly 115 proximate the distal end of implant 31, such as to allow a repeated pullback imaging procedure to be performed). In some embodiments, spring tip 119 s comprises a varying flexibility along its length, for example, when spring tip 119s is more flexible at its distal end than at its proximal end. In some embodiments, the proximal end of spring tip 119s comprises a flexibility approximately equal to the flexibility of the distal end of shaft 120 of imaging probe 100, and the distal end of spring tip 119s comprises a flexibility less than the flexibility of the distal end of shaft 120.

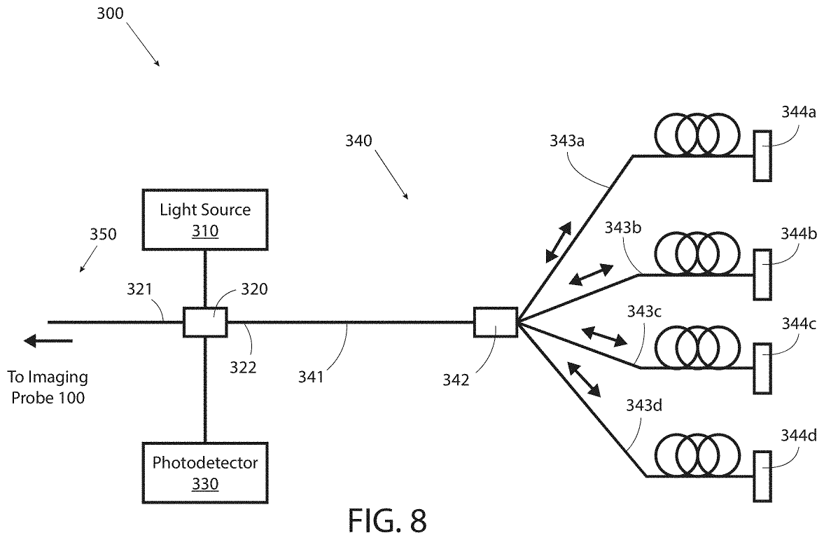

Referring now to FIG. 8, a schematic view of a portion of an imaging assembly is illustrated, consistent with the present inventive concepts. Imaging assembly 300 includes a balanced interferometer, interferometer 320, which is optically coupled to both light source 310 and a light measurement element, photodetector 330. Interferometer 320 includes a reference arm, arm 322, which is optically coupled to a reference pathway, pathway 340, and a sample arm, arm 321. Sample arm 321 is optically coupled to a sample pathway, pathway 350, that includes the optical path of an attached imaging probe 100. Interferometer 320 can be configured to receive light transmitted from light source 310 and it can optically split the received light to further transmit the light along both reference pathway 340 and sample pathway 350. The light that propagates away from interferometer 320 along each pathway 340,350, is reflected back along each pathway, and the reflections are received by interferometer 320. Interferometer 320 then directs the reflected light received from each pathway 340,350 to photodetector 330. Photodetector 330 produces a signal related to the reflected light (e.g. a signal related to the difference in phase, amplitude, polarization, a spectral difference, or any combination of these, in the light received from each pathway 340,350). The signal produced by photodetector 330 is used by system 10 to generate an OCT image, for example, when processor 52 comprises a digital signal processor, which can be configured to process the signal and generate the OCT image.

The axial resolution of an OCT image is strongly dependent on the matched optical properties of reference pathway 340 and sample pathway 350. For example, the optical dispersion of the optical fibers of pathways 340,350 should be approximately the same (e.g. the average dispersion along the length of each pathway) for the optimal OCT image resolution. In the illustrated embodiment, imaging assembly 300 incudes multiple reference paths optically connected to interferometer 320 via an optical switch, switch 342. Switch 342 is optically coupled to interferometer 320 via a fiber, fiber 341. The multiple reference paths each comprise a length of fiber, for example, four fibers 343a-d shown, where each fiber 343 is optically coupled to a mirror, for example, mirrors 344a-d shown. Each fiber 343a-d can comprise a different optical dispersion, for example, to cover a range of dispersion values likely to match the optical dispersion of imaging probe 100. Switch 342 is configured to optically connect one of the multiple reference path fibers 343 to interferometer 320. Switch 342 can be configured to switch between the multiple fibers 343 to connect the fiber that best matches the dispersion of a connected imaging probe 100. It can be difficult and/or expensive to mass produce imaging probes 100 each comprising an optical dispersion within a narrow range of acceptable values. By enabling a wider range of dispersion values for imaging probes 100, imaging assembly 300 can accommodate a more cost-effective manufacturing of the various probes 100. In some embodiments, console 50 can perform a calibration process to match a connected imaging probe 100 (e.g. a probe with an unknown dispersion value) to the appropriate reference path. This calibration can be performed each time a probe 100 is operably connected to console 50.

Referring now to FIGS. 9A and 9B, various views of portions of an imaging probe are illustrated, consistent with the present inventive concepts. In FIG. 9A, the distal portion of imaging probe 100 is illustrated. Shaft 120 surrounds core 110 and optical assembly 115. Distal tip 119 comprises a spring tip extending from the distal end of shaft 120. In some embodiments, distal tip 119 comprises one or more anchoring elements, retainer 1191. Retainer 1191, as shown, extends proximally from spring tip 119 and is anchored within the distal end of shaft 120. Retainer 1191 can be fixedly attached to the coil of spring tip 119 via a wire, core wire 1192.

Optical assembly 115 can be positioned at the distal end of optical core 110. Optical assembly 115 can comprise a lens assembly, assembly 1151, which is optically and physically coupled to the distal end of optical core 110. Lens assembly 1151 can comprise a GRIN lens comprising a beveled distal end. The beveled distal end of lens assembly 1151 can comprise a total internally reflective surface. Optical assembly 115 can comprise lens marker 1156, an element that can be imaged by a separate imaging device. Marker 1156 can comprise a radiopaque marker configured to be imaged using fluoroscopy or other X-ray based imaging device. In some embodiments, marker 1156 comprises a coil helically wrapped about a distal portion of optical core 110. In some embodiments, marker 1156 abuts the proximal end of lens assembly 1151 (e.g. marker 1156 is proximate the splice between lens assembly 1151 and optical core 110). In some embodiments, marker 1156 comprises an outer diameter approximately equal to the outer diameter of lens assembly 1151. In some embodiments, marker 1156 is positioned in direct contact with optical core 110 (e.g. in direct contact with the glass surface of optical core 110). In some embodiments, marker 1156 comprises a pitch at least 1.5 times greater than the diameter of the wire of marker 1156, such that marker 1156 comprises a spacing between the coils of at least half of the width of the wire of marker 1156. This spacing can increase the flexibility of marker 1156, such as to prevent stress concentrations in optical core 110 (e.g. stress concentrations at the proximal and/or distal end of marker 1156 that may be at an undesirable high level if marker 1156 is too stiff). In some embodiments, marker 1156 is adhered to optical core 110 using an adhesive. In some embodiments, the spacing between coils of marker 1156 is sufficient to allow an adhesive to wick into the coils of marker 1156, providing a uniform distribution of the adhesive about marker 1156. In some embodiments, marker 1156 is adhered using an adhesive that is visible under UV light. In some embodiments, in manufacturing, UV light can be used to inspect marker 1156 to make sure the adhesive has been properly applied and distributed about the marker 1156 (e.g. about the coils of marker 1156). In some embodiments, the adhesive used to adhere marker 1156 is also configured to provide support to the splice between optical core 110 and lens assembly 1151.

An elongate tube, tube 1154, can surround at least a distal portion of optical core 110, lens assembly 1151, and a sealing element, plug 1153. In some embodiments, tube 1154 surrounds at least a portion of marker 1156. Tube 1154 can comprise a heat shrink material. Tube 1154 can comprise PET. At least a portion of tube 1154 can be adhesively attached, or otherwise attached, to at least a portion of lens assembly 1151, optical core 110, and/or plug 1153. Plug 1153 can be configured to prevent and/or at least limit the egress of gel 118 into a cavity created between lens assembly 1151 and plug 1153, space 1152 shown. Space 1152 can be filled with air and/or one or more other fluids. The fluid within space 1152 can be configured to provide desired optical properties between lens assembly 1151 and the fluid (e.g. configured to provide a glass-air interface).

FIG. 9B shows a perspective view of optical assembly 115 and the distal portion of optical core 110. In some embodiments, plug 1153 comprises a smaller outer diameter than lens assembly 1151. In some embodiments, tube 1154 is heat shrunk onto both lens assembly 1151 and plug 1153, such that tube 1154 is arranged in a profile similar to that which is illustrated in FIG. 9B, such that space 1152 comprises a variable outer diameter, and at least a portion of that diameter is less than the outer diameter of lens assembly 1151. In some embodiments, tube 1154 does not come into contact with the beveled distal end of lens assembly 1151, for example, such that only the fluid within space 1152 contacts the distal end of lens assembly 1151, ensuring a total internal reflection of the distal end of lens assembly 1151 (e.g. due to the glass-air interface at the distal end of lens assembly 1151). In some embodiments, the reduced diameter of plug 1153 allows imaging probe 100 to achieve a tighter (i.e. smaller) bend radius than would be achievable if plug 1153 comprised the same or a larger outer diameter as lens assembly 1151.

In some embodiments, plug 1153 comprises a sintered construction, such as a plug comprising sintered stainless steel. Plug 1153 can comprise an outer diameter of approximately 0.020″. Plug 1153 can comprise 316L stainless steel. In some embodiments, plug 1153 can comprise a porosity sufficient to allow gas to pass through plug 1153. Additionally, the porosity of plug 1153 can prevent the passage of viscous liquids, for example liquids with a viscosity greater than 10.

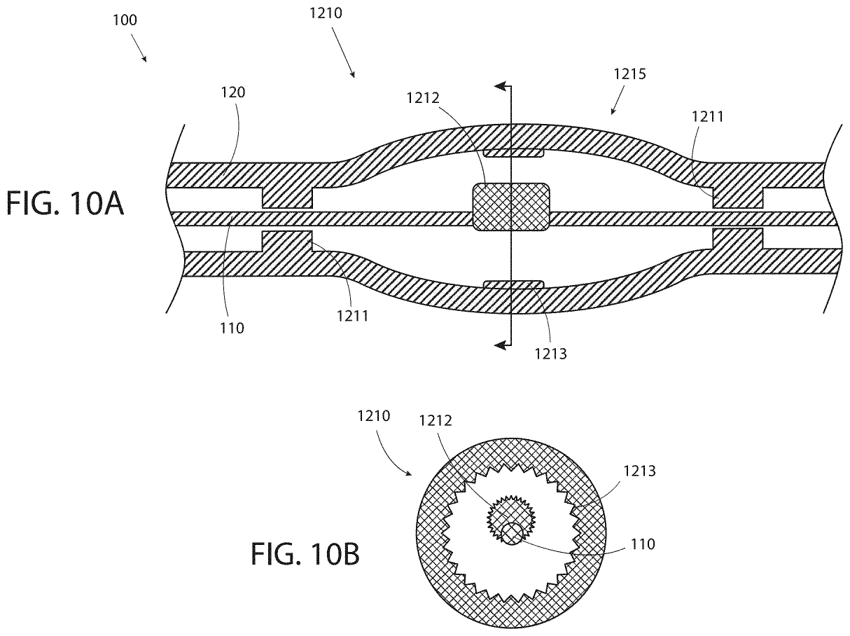

Referring now to FIGS. 10A and 10B, sectional views of an unbalanced centrifugal breaking assembly are illustrated, consistent with the present inventive concepts. In the event that rotation assembly 500 (described herein) were to rotate optical core 110 too fast (e.g. in a failure mode where core 110 is spun above a safety threshold rate of rotation), a mechanical stopping mechanism could help to ensure patient safety. FIGS. 10A and 10B illustrate a portion of shaft 120 of imaging probe 100 comprising a mechanical mechanism, break assembly 1210, constructed and arranged to prevent the distal portion (e.g. at least the patient inserted portion) of optical core 110 from spinning at a speed above a safety threshold. Imaging probe 100 can be of similar construction and arrangement and comprise similar components to probe 100 described in reference to FIGS. 1, 1A, and 1B, and as otherwise described herein. Break assembly 1210 can comprise an expanded portion of shaft 120, chamber 1215, through which optical core 110 rotates. Shaft 120 can include one or more bearing surfaces, bearings 1211, for example bearings 1211 positioned proximal and distal to chamber 1215, configured to center core 110 within chamber 1215 as it rotates. Break assembly 1210 can comprise an eccentric mass attached to core 110, weight 1212. Weight 1212 can be attached to core 110 and configured to offset the axis of rotation of core 110 as it rotates. In some embodiments, the inner surface of chamber 1215 comprises a textured surface, such as a gear-like surface comprising one or more teeth, teeth 1213. As core 110 spins, weight 1212 is pulled towards the inner surface of chamber 1215 via centrifugal force. This force acts against the stiffness of core 110, which is held in the center of chamber 1215 via bearings 1211. Once optical core 110 rotates at a rate above a threshold rate of rotation, the centrifugal force will overcome the stiffness of imaging probe 100 enough that weight 1212 will contact teeth 1213. In some embodiments, this contact will cause optical core 110 to break, thereby disconnecting the distal portion of optical core 110 from the rotary force provided by rotation assembly 500 from the proximal end. Alternatively or additionally, contact between weight 1212 and the inner surface of chamber 1215 can provide other means of stopping rotation of core 110, for example, by closing an electrical circuit configured to cut power to rotation assembly 500, or by causing a locking force configured to stall rotation assembly 500.

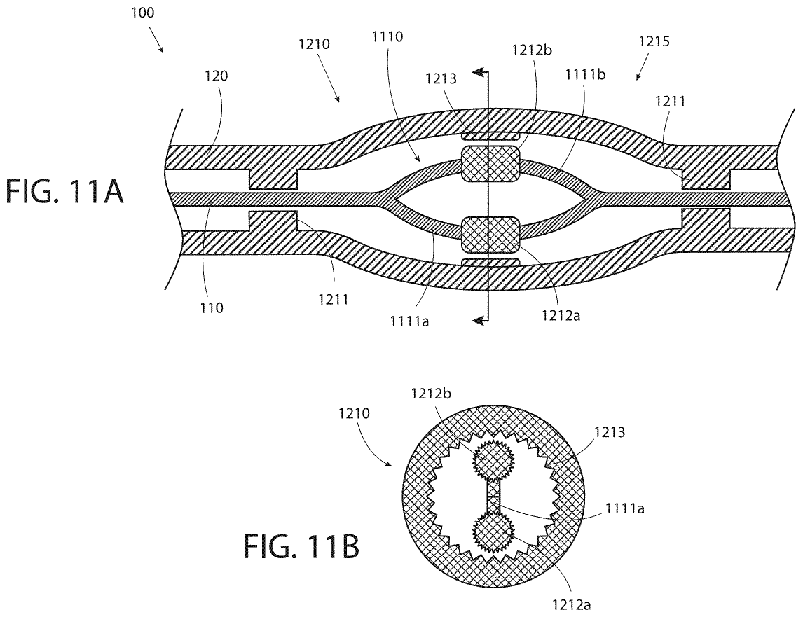

Referring additionally to FIGS. 11A and 11B, sectional views of a balanced centrifugal breaking assembly are illustrated, consistent with the present inventive concepts. In some embodiments, optical core 110 can be split into two portions, bifurcation 1110, comprising a first arm 1111a and second arm 1111b. Bifurcation 1110 can be positioned within a portion of optical core 110, such that optical core 110 is unitary proximal to and distal to bifurcation 1110 as shown. Bifurcation 1110 can be positioned within chamber 1215 of break assembly 1210. In some embodiments, each arm 1111a,b of bifurcation 1110 comprises a mass attached thereto, weights 1212a and 1212b, respectively. In this balanced design, weights 1212a,b are balanced during rotation of optical core 110, limiting vibrational forces caused by break assembly 1210. Under normal operating speeds, the stiffness of bifurcation 1110 prevents weights 1212a,b from contacting teeth 1213. When core 110 spins above a threshold, the centrifugal force overcomes the stiffness of bifurcation 1110 and weights 1212a,b contact teeth 1213, causing the distal end of optical core 110 to stop rotating as described herein.

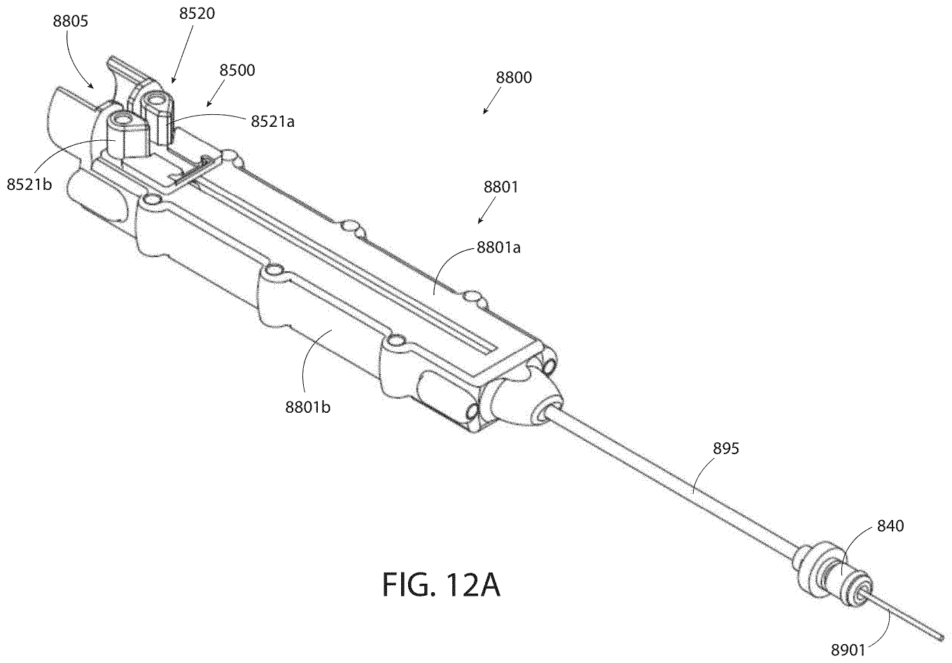

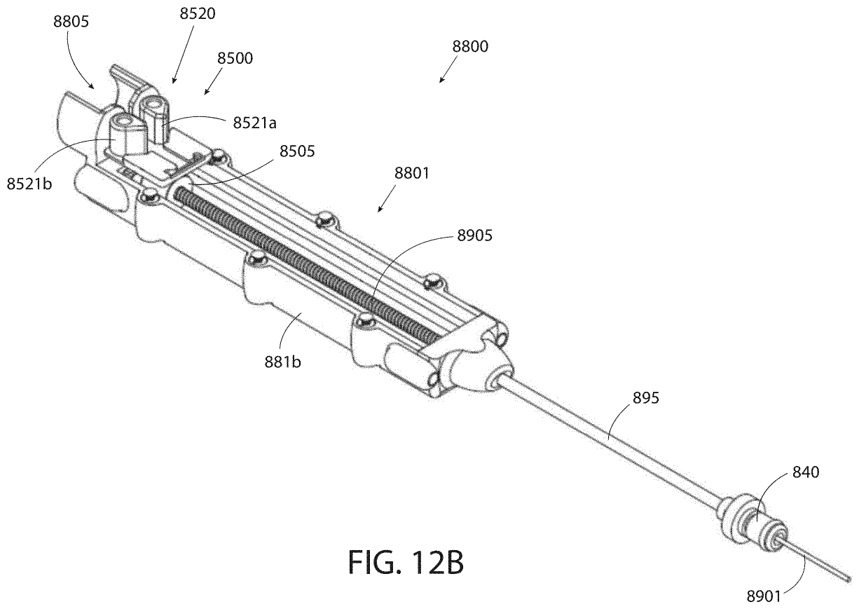

Referring now to FIGS. 12A and 12B, perspective views of a pullback module are illustrated, consistent with the present inventive concepts. Pullback module 8800 of FIGS. 12A and 12B can be of similar construction and arrangement to pullback module 880 described in FIGS. 1A, 2A, and as otherwise described herein. Pullback module 8800 can include a structure surrounding one or more components of the module, housing 8801. In some embodiments, pullback module 8800 can comprise a disposable module, for example, a module configured to be used in a single clinical procedure. Housing 8801 can comprise a multipart housing, for example, shell 8801b and cover 8801a. In FIG. 12B, cover 8801a has been removed for illustrative clarity. Pullback module 8880 can be configured to removably attach to a portion of delivery catheter 80, as well as shaft 120 of imaging probe 100, and to retract imaging probe 100 relative to delivery catheter 80. In some embodiments, a linkage 8901 extends through a conduit, sheath 895 from motive element 830 (described herein) to pullback module 8800. Pullback module 8800 can comprise a linear drive assembly, for example, a linear drive assembly including a lead screw 8905 extending longitudinally through housing 8801. Lead screw 8905 can be operably attached to linkage 8901, such that when linkage 8901 is rotated (e.g. via motive element 830), lead screw 8905 rotates in unison. In some embodiments, linkage 8901 comprises a torque cable. In some embodiments, linkage 8901 and/or the interior surface of sheath 895 comprises a coating configured to minimize the frictional force between these components (e.g. while linkage 8901 is rotating within sheath 895).

Pullback module 8800 can comprise a retraction assembly, puller 8500. Puller 8500 can be of similar construction and arrangement to puller 850 described herein. Puller 8500 can be constructed and arranged to releasably attach to shaft 120 of imaging probe 100. In some embodiments, puller 8500 comprises a locking mechanism, cam lock 8520. Cam lock 8520 can comprise a unidirectional locking mechanism comprising a pair of cams, cam 8521a and 8521b, configured to frictionally engage shaft 120 when puller 8500 is retracted. Housing 8801 can include a retaining mechanism, port 8805, constructed and arranged to removably attach to delivery catheter 80, for example, to connector 82 of delivery catheter 80 (described herein). Pullback module 8800 can be configured to affix to connector 82, and to pullback imaging probe 100 relative to delivery catheter 80, for example, as puller 8500 retracts and cam lock 8520 engages shaft 120. Puller 8500 can include an engagement mechanism, nut 8505, configured to rotatably engage lead screw 8905, such that as lead screw 8905 rotates, nut 8505 translates along screw 8905. In some embodiments, nut 8505 is configured to engage in a first direction, and to slip in an opposite direction, such that puller 8500 can be manually advanced (e.g. towards port 8805) and retracted when screw 8905 is rotated.

The above-described embodiments should be understood to serve only as illustrative examples; further embodiments are envisaged. Any feature described herein in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the invention, which is defined in the accompanying claims.

What is claimed is:

1. An imaging system for a patient comprising:
an imaging probe, comprising:
an elongate shaft comprising a proximal end, a distal portion, and a lumen extending between the proximal end and the distal portion;
a rotatable optical core comprising a proximal end and a distal end, wherein at least a portion of the rotatable optical core is positioned within the lumen of the elongate shaft; and
an optical assembly positioned proximate the distal end of the rotatable optical core, the optical assembly configured to direct light to tissue to be imaged, and to collect reflected light from the tissue to be imaged; and
an imaging assembly constructed and arranged to optically couple to the imaging probe, the imaging assembly configured to emit light into the imaging probe and receive the reflected light collected by the optical assembly,
wherein the imaging assembly comprises multiple reference paths, wherein each reference path comprises an optical fiber that comprises a different optical dispersion, and wherein the imaging assembly is constructed and arranged to select a reference path that matches the optical dispersion of the rotatable optical core.

2. The system according to claim 1, wherein the elongate shaft further comprises an optically transparent window including one or more imagable portions, and wherein the system further comprises an algorithm configured to utilize image data provided by the one or more imagable portions to reduce the negative impact of NURD on the one or more produced images.

3. The system according to claim 1, further comprising an algorithm, wherein the algorithm is configured to modify different image data portions of the one or more produced images based on one, two, or more characteristics of those image data portions.

4. The system according to claim 3, wherein the algorithm is configured to exponentially increase the intensity of an image data portion based on the distance of the portion from the center of the produced image.

5. The system according to claim 3, wherein the algorithm is configured to compensate the image data portion based on the physical, optical, and or other properties of the imaging system.

6. The system according to claim 1, further comprising a light source, wherein the imaging assembly is constructed and arranged to receive light from the light source, and wherein the imaging assembly is constructed and arranged to duplicate and shift the light received from the light source.

7. The system according to claim 6, wherein the light emitted into the imaging probe comprises a duty cycle that is two times the duty cycle of the light received from the light source.

8. The system according to claim 1, wherein the imaging probe further comprises a spring tip comprising a varying flexibility along its length, and wherein during a pullback procedure the spring tip is constructed and arranged to remain distal at a pullback starting location of the optical assembly.

9. The system according to claim 1, wherein the optical assembly comprises a lens and an air-filled space distal to the lens, and wherein the air-filled space is sealed with a porous plug comprising a sintered construction.

10. The system according to claim 1, wherein the imaging probe comprises a centrifugal breaking assembly constructed and arranged to prevent the optical assembly from rotating above a threshold rate of rotation.

11. The system according to claim 10, wherein the centrifugal breaking assembly comprises an unbalanced centrifugal breaking assembly.

12. They system according to claim 10, wherein the centrifugal breaking assembly comprises a balanced centrifugal breaking assembly.

13. The system according to claim 1, further comprising a pullback module comprising a unidirectional locking mechanism constructed and arranged to frictionally engage the elongate shaft of the imaging probe, and a lead screw mechanism constructed and arranged to pull back the imaging probe via the locking mechanism.
